(19) United States
(12) Patent Application Publication
(10) Pub. No.: US 2013/0109633 A1
(43) Pub. Date: May 2, 2013

(54) CB-183,315 Compositions and Related Methods

(71) Applicants: Sandra O'Connor, Hudson, NH (US); Sophie Sun, Lexington, MA (US); Gaauri Naik, Cambridge, MA (US)

(72) Inventors: Sandra O'Connor, Hudson, NH (US); Sophie Sun, Lexington, MA (US); Gaauri Naik, Cambridge, MA (US)

(73) Assignee: Cubist Pharmaceuticals, Inc., Lexington, MA (US)

(21) Appl. No.: 13/481,009

(22) Filed: May 25, 2012

Related U.S. Application Data
(60) Provisional application No. 61/490,584, filed on May 26, 2011.

(51) Int. Cl. A61K 38/12

(52) U.S. Cl. CPC A61K 38/12
USPC 514/21.1

(57) ABSTRACT

The present disclosure provides novel solid CB-183,315 formulations which have improved chemical stability. The chemical stability of the solid CB-183,315 is dependent on the process by which the composition is made. Solid preparations of CB-183,315 can be prepared by the following method: (a) forming an aqueous solution of CB-183,315 and at least one sugar that (e.g., sucrose, trehalose or dextran) at a pH of 2-7, preferably pH 6 and (b) converting the aqueous solution to the solid preparation of CB-183,315 (e.g., via lyophilization or spray drying).

### RELATED APPLICATIONS

This application claims the benefit of U.S. provisional patent application No. 61/490,584, filed on May 26, 2011, which is incorporated herein by reference in its entirety.

### TECHNICAL FIELD

The present invention relates to solid CB-183,315 preparations, pharmaceutical compositions comprising the solid CB-183,315 preparations, as well as methods of making the solid CB-183,315 preparations. Preferred improved compositions include solid CB-183,315 preparations with increased CB-183,315 stability.

### BACKGROUND

CB-183,315 is a cyclic lipopeptide antibiotic currently in Phase III clinical trials for the treatment of Clostridium difficile-associated disease (CDAD). As disclosed in International Patent Application WO 2010/075215, herein incorporated by reference in its entirety, CB-183,315 has antibacterial activity against a broad spectrum of bacteria, including drug-resistant bacteria and C. difficile. Further, the CB-183,315 exhibits bacteriacidal activity.

CB-183,315 (FIG. 1) can be made by the deacylation of BOC-protected daptomycin, followed by acylation and deprotection as described in International Patent Application WO 2010/075215.

During the preparation and storage of CB-183,315, the CB-183,315 molecule can convert to structurally similar compounds as shown in FIGS. 2-4, leading to the formation of anhydro-CB-183,315 (FIG. 3) and beta-isomer of CB-183,315 (“B-isomer CB183,315” in FIG. 2). Accordingly, one measure of the chemical stability of CB-183,315 is the amount of CB-183,315 (FIG. 1) present in the CB-183,315 composition relative to the amount of structurally similar compounds including anhydro-CB-183,315 (FIG. 3) and beta-isomer of CB-183,315 (FIG. 2). The amount of CB-183,315 relative to the amount of these structurally similar compounds can be measured by high performance liquid chromatography (HPLC) after reconstitution in an aqueous diluent (e.g., as described in Example 10). In particular, the purity of CB-183,315 and amounts of structurally similar compounds (e.g., FIGS. 2, 3 and 4) can be determined from peak areas obtained from HPLC (e.g., according to Example 10 herein), and measuring the rate of change in the amounts of CB-183,315 over time can provide a measure of CB-183,315 chemical stability in a solid form.

There is a need for solid CB-183,315 compositions with improved chemical stability in the solid form (i.e., higher total percent CB-183,315 purity over time), providing advantages of longer shelf life, increased tolerance for more varied storage conditions (e.g., higher temperature or humidity) and increased chemical stability.

### SUMMARY

The present invention provides CB-183,315 compositions with improved CB-183,315 chemical stability, measured as a higher total percent CB-183,315 purity over time (as determined by HPLC according to the method of Example 10). Surprisingly, the CB-183,315 contained in solid preparations with certain preferred compositions, for example, in compositions with certain sugars (e.g., CB-183,315 combined with sucrose or trehalose) was more chemically stable than CB-183,315 in CB-183,315 solid preparations without sugar. Even more surprising was that the chemical stability of the solid CB-183,315/sugar formulations was dependent on the process by which the composition was made. Solid preparations of CB-183,315 can be prepared by the following method: (a) forming an aqueous solution of CB-183,315 and at least one sugar (e.g. sucrose, trehalose or trehalose combined with dextran), at a pH of 2-7, preferably pH 2-6 and most preferably about 6 and (b) converting the aqueous solution to the solid CB-183,315/sugar preparation (e.g via lyophilization or spray drying). The chemical stability of CB-183,315 in a solid form was measured by comparing total CB-183,315 purity measurements from multiple solid CB-183,315 preparations each obtained according to Example 10. Higher chemical stability was measured as higher comparative CB-183,315 total purity measurements between two samples according to Example 10.

Preferred examples of solid pharmaceutical CB-183,315 preparations include a ratio (w/w) of about at least 1:0.3 to about 1:3 of CB-183,315 to one or more non-reducing sugars. Other preferred examples of solid pharmaceutical CB-183,315 preparations include a ratio (w/w) of about at least 1:0.5 to about 1:2, more preferably about 1:1 of CB-183,315 to one or more non-reducing sugars.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 shows the chemical structures of CB-183,315.

FIG. 2 shows the beta isomer of CB-183,315 (“one component, RS-3b of Impurity RS-3ab”).

FIG. 3 shows the anhydro-CB-183,315 (“Impurity RS-6”).

FIG. 4 shows the proposed structure of RS-3a, which co-elutes with Impurity RS-3b.

FIG. 5A is a graph showing the percent increase of impurity RS-6 in CB-183,315 formulations (no sugar) formulated at varying pH ranges designated Formulations A, B, C and D measured as a function of time at 40 degrees C. (as described in Example 10). The parenthetical numbers in the legend represent the weight percent of moisture present in the sample as measured by Karl Fischer titration.

FIG. 5B is a graph showing the percent increase of impurity RS-3ab in CB-183,315 formulations (no sugar) formulated at varying pH ranges designated Formulations A, B, C and D measured as a function of time at 40 degrees C. (as described in Example 10). The parenthetical numbers in the legend represent the weight percent of moisture present in the sample as measured by Karl Fischer titration.

FIG. 6A is a graph showing the percent increase of impurity RS-6 in CB-183,315/sucrose formulations formulated at pH 3-4 with varying sucrose concentrations designated Formulations E, F and G and comparative Formulation A (CB-183,315 no sugar) measured as a function of time at 40 degrees C. (as described in Example 10). The parenthetical numbers in the legend represent the weight percent of moisture present in the sample as measured by Karl Fischer titration.

FIG. 6B is a graph showing the percent increase of Impurity RS-3ab in CB-183,315/sucrose formulations formulated at pH 3-4 with varying sucrose concentrations designated Formulations E, F and G and comparative Formulation A (CB-183,315 no sugar) measured as a function of time at 40 degrees C. (as described in Example 10). The parenthetical numbers in the legend represent the weight percent of moisture present in the sample as measured by Karl Fischer titration.

FIG. 7A is a graph shoving the percent increase of Impurity RS-6 in CB-183,315/sucrose (1:1.5 w/w) formulations formulated at varying pH designated Formulations G, H, I, J, K and L measured as a function of time at 40 degrees C. (as described in Example 10). The parenthetical numbers in the legend represent the weight percent of moisture present in the sample as measured by Karl Fischer titration.

FIG. 7B is a graph showing the percent increase of impurity RS-3ab in CB-183,315/sucrose (1:1.5 w/w) formulations formulated at varying pH designated Formulations G, H, I, J, K and L measured as a function of time at 40 degrees C. (as described in Example 10). The parenthetical numbers in the legend represent the weight percent of moisture present in the sample as measured by Karl Fischer titration.

FIG. 8A is a graph showing the percent increase of Impurity RS-6 in CB-183,315/sucrose formulations formulated at pH 6 with varying sucrose concentrations designated Formulations J and M and comparative Formulation C (CB-183,315 no sugar) measured as a function of time at 40 degrees C. (as described in Example 10). The parenthetical numbers in the legend represent the weight percent of moisture present in the sample as measured by Karl Fischer titration.

FIG. 8B is a graph showing the percent increase of Impurity RS-3ab in CB-183,315/sucrose formulations formulated at pH 6 with varying sucrose concentrations designated Formulations J and M and comparative Formulation C (CB-183,315 no sugar) measured as a function of time at 40 degrees C. (as described in Example 10). The parenthetical numbers in the legend represent the weight percent of moisture present in the sample as measured by Karl Fischer titration.

FIG. 9A is a graph showing the percent increase of Impurity RS-6 in preferred CB-183,315/sucrose formulation designated Formulation Q and Comparator formulations designated Formulations O, P and N measured as a function of time at 40 degrees C. (as described in Example 10). The parenthetical numbers in the legend represent the weight percent of moisture present in the sample as measured by Karl Fischer titration.

FIG. 9B is a graph showing the percent increase of Impurity RS-3ab in preferred CB-183,315/sucrose formulation designated Formulation Q and comparator formulations designated Formulations O, P and N measured as a function of time at 40 degrees C. (as described in Example 10). The parenthetical numbers in the legend represent the weight percent of moisture present in the sample as measured by Karl Fischer titration.

FIG. 9C is a graph showing the percent decrease of CB-183,315 in preferred CB-183,315/sucrose formulation designated Formulation Q and comparator formulations designated Formulations O, P and N measured as a function of time at 40 degrees C. (as described in Example 10). The parenthetical numbers in the legend represent the weight percent of moisture present in the sample as measured by Karl Fischer titration.

FIG. 10A is a graph showing the percent increase of Impurity RS-6 in CB-183,315/sucrose formulations designated Formulations R, S and T and Comparator formulation designated Formulation C measured as a function of time at 40 degrees C. (as described in Example 10). The parenthetical numbers in the legend represent the weight percent of moisture present in the sample as measured by Karl Fischer titration.

FIG. 10B is a graph showing the percent increase of Impurity RS-3ab in CB-183,315/sucrose formulations designated Formulations R, S and T and Comparator formulation designated Formulation C measured as a function of time at 40 degrees C. (as described in Example 10). The parenthetical numbers in the legend represent the weight percent of moisture present in the sample as measured by Karl Fischer titration.

FIG. 10C is a graph showing the percent decrease of CB-183,315 in CB-183,315/sucrose formulations designated Formulations R, S and T and Comparator formulations designated Formulation C measured as a function of time at 40 degrees C. (as described in Example 10). The parenthetical numbers in the legend represent the weight percent of moisture present in the sample as measured by Karl Fischer titration.

FIG. 11A is a graph showing the percent increase of Impurity RS-6 in preferred CB-183,315/sucrose formulations designated Formulations Q, U and R and Comparator formulation designated Formulation N measured as a function of time at 40 degrees C. (as described in Example 10). The parenthetical numbers in the legend represent the weight percent of moisture present in the sample as measured by Karl Fischer titration.

FIG. 11B is a graph showing the percent increase of Impurity RS-3ab in preferred CB-183,315/sucrose formulations designated Formulations Q, U and R and Comparator formulation designated Formulation N measured as a function of time at 40 degrees C. (as described in Example 10). The parenthetical numbers in the legend represent the weight percent of moisture present in the sample as measured by Karl Fischer titration.

FIG. 11C is a graph showing the percent decrease of CB-183,315 in preferred CB-183,315/sucrose formulations designated Formulations Q, U and R and Comparator formulation designated Formulation N measured as a function of time at 40 degrees C. (as described in Example 10). The parenthetical numbers in the legend represent the weight percent of moisture present in the sample as measured by Karl Fischer titration.

FIG. 12A is a graph showing the percent increase of Impurity RS-6 in preferred formulations designated Formulations Q and M and comparative formulation designated Formula C measured as a function of time at 25 degrees C. (as described in Example 10). The parenthetical numbers in the legend represent the weight percent of moisture present in the sample as measured by Karl Fischer titration.

FIG. 12B is a graph showing the percent increase of Impurity RS-3ab in preferred formulations designated Formulations Q and M and comparative formulation designated Formula C measured as a function of time at 25 degrees C. (as described in Example 10). The parenthetical numbers in the legend represent the weight percent of moisture present in the sample as measured by Karl Fischer titration.

FIG. 12C is a graph showing the percent decrease of CB-183,315 in preferred formulations designated Formulations Q and M and comparative formulation designated Formula C measured as a function of time at 25 degrees C. (as described in Example 10). The parenthetical numbers in the legend represent the weight percent of moisture present in the sample as measured by Karl Fischer titration.

FIG. 13A is a graph showing the percent increase of Impurity RS-6 in preferred formulations designated Formulations Q and M and comparative formulation designated Formula C measured as a function of time at 40 degrees C. (as described in Example 10). The parenthetical numbers in the legend represent the weight percent of moisture present in the sample as measured by Karl Fischer titration.

FIG. 13B is a graph showing the percent increase of Impurity RS-3ab in preferred formulations designated Formulations Q and M and comparative formulation designated Formula C measured as a function of time at 40 degrees C. (as described in Example 10). The parenthetical numbers in the legend represent the weight percent of moisture present in the sample as measured by Karl Fischer titration.

FIG. 13C is a graph showing the percent decrease of CB-183,315 in preferred formulations designated Formulations Q and M and comparative formulation designated Formula C measured as a function of time at 40 degrees C. (as described in Example 10). The parenthetical numbers in the legend represent the weight percent of moisture present in the sample as measured by Karl Fischer titration.

### DETAILED DESCRIPTION

The present invention is based in part on the unexpected discovery that combining CB-183,315 in solution with one or more sugars (e.g., sucrose or trehalose) and then converting the solution to a solid form (e.g., by lyophilization or spray drying) provides a solid composition with increased CB-183,315 chemical stability. Preferred CB-183,315 pharmaceutical composition's include pharmaceutical compositions formulated for oral delivery, obtained by combining these solid forms with one or more excipients.

The term “CB-183,315/sugar” refer to the CB-183,315 solid preparation comprising the composition that arises from combining CB-183,315 in solution with one or more sugars (e.g., sucrose or trehalose) and then converting the solution to a solid form (e.g., by lyophilization or spray drying). The terms “CB-183,315/sucrose”, “CB-183,315/trehalose” and the like refer to CB-183,315 solid composition comprising the composition that arises from combining CB-183,315 in solution with one or more particular sugars (e.g., sucrose or trehalose) and then converting the solution to a solid form (e.g., by lyophilization or spray drying). CB-183,315/sugar may also contain excipients, fillers, adjuvents, stabilizers and the like.

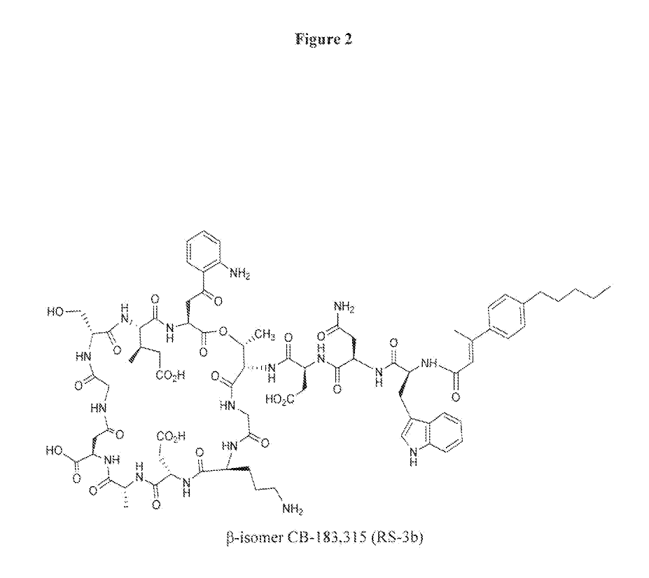

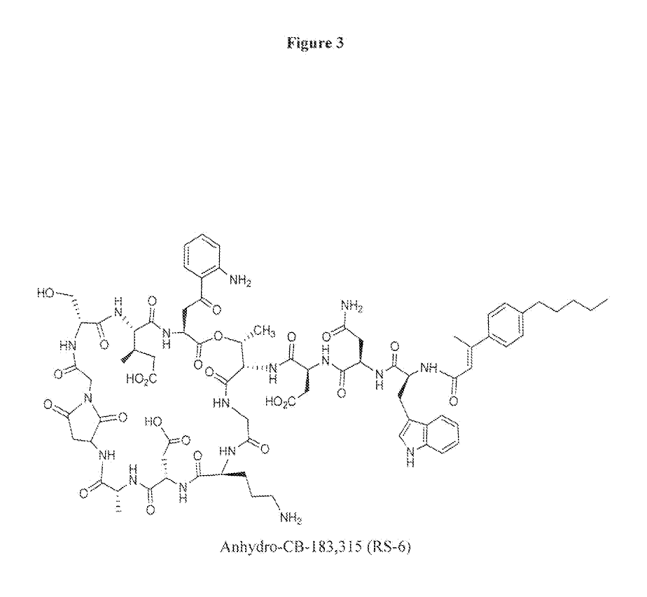

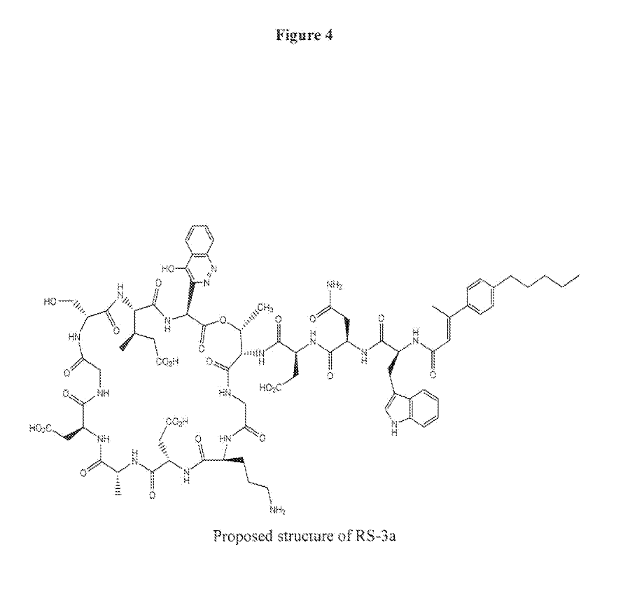

CB-183,315 chemical stability refers to the change in the measured CB-183,315 purity measured by high performance liquid chromatography (HPLC). The change in CB-183,315 purity can be determined by measuring and comparing the amount(s) of CB-183,315 and/or structurally similar compounds (FIGS. 2, 3 and 4) in samples taken from a solid composition over a period of time. The chemical stability of CB-183,315 in the solid form or pharmaceutical compositions containing CB-183,315 was measured by measuring the amount of CB-183,315, as well as the amount of the structurally similar compounds anhydro-CB-183,315 (FIG. 3) and the mixture of co-eluted compounds, beta-isomer of CB-183,315 (FIG. 2) and RS-3a (FIG. 4), collectively known as “RS-3ab”, present in a solid form using the HPLC method described in Example 10. Solid forms of CB-183,315 obtained by lyophilizing or spray drying solutions of CB-183,315 with one or more sugars (e.g., Table 1 Formulations E-M, and Q-U) demonstrated a higher stability than solid forms of CB-183,315 obtained by lyophilizing or spray drying CB-183,315 without a sugar (e.g., Formulations A-D, and N Table 1). CB-183,315 stability was measured by the HPLC method of Example 10 showing a slower reduction in the amount of (or greater amounts of) CB-183,315 in the more stable solid forms (e.g Formulations Q-U Table 1) than in the comparative formulations (CB-183,315 e.g., Formulations A-D and N Table 1). Solid forms of CB-183,315 with higher stability also showed slower rates of increase (or lower amounts of) anhydro-CB-183,315 (FIG. 3) and/or the mixture of co-eluted compounds, beta-isomer of CB-183,315 (FIG. 2) and RS-3a (FIG. 4), collectively known as “RS-3ab” measured over time in the solid form by the HPLC method of Example 10.

Solid pharmaceutical CB-183,315/sugar preparation having increased CB-183,315 stability can be obtained by converting a solution containing CB-183,315 and a sugar to a solid form. The solution can be an aqueous solution containing one or more sugars (preferably a non-reducing sugar such as sucrose or trehalose) in an amount effective to decrease the amount of substances selected from the group consisting of the anhydro-CB-183,315 (FIG. 3), and/or the beta-isomer of CB-183,315 (FIG. 2), as measured by the HPLC method of Example 10 in the resulting solid form. The solution can include CB-183,315 and a sugar in an amount effective to increase the chemical stability of CB-183,315. Preferred examples of solid CB-183,315 preparations include a ratio of about at least 1:0.3 to about 1:3 of CB-183,315 to one or more non-reducing sugars (w/w). Examples of CB-183,315 to one or more non-reducing sugars (w/w) ratios include about 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, and about 3:1. As described in Examples 2, 6 and 7, solid CB-183-315 compositions with increased CB-183,315 chemical stability include a non-reducing sugar (e.g., such as sucrose or trehalose) or a combination of non-reducing sugars (e.g., sucrose and trehalose). The solution can be formed by dissolving CB-183,315 in water, dissolving the sugar in the aqueous CB-183,315 solution, and adjusting the solution to a suitable pH. The pH is selected to provide a solution that, when converted to a solid form, is characterized by increased CB-183,315 stability (e.g., higher measured amounts of CB-183,315 over time, and/or lower measured amounts of Impurity RS-6 and/or lower measured amounts of Impurity RS-3ab). For example, the pH of the solution can be about 2-7. The pH can be about 1, 2, 3, 4, 5, 6 or 7, preferably about 2-6, 3-6, 3.5-6, and most preferably about 6. The solution comprising CB-183,315 and the sugar(s) can be converted to a solid form by any suitable method. For example, the solution can be lyophilized (e.g., Example 9), spray dried (e.g., Example 8), fluid bed dried, crystallized, spray congealed or spray layered.

A preferred method of making a solid CB-183,315 preparation comprises
a. forming an aqueous solution comprising CB-183,315 and a sugar selected from sucrose or trehalose, wherein the CB-183,315 to sugar is present in a range of about at least 1:0.5 to about 1:2 by weight, at a pH of about 2-7, and
b. converting the aqueous CB-183,315 of step (a) to the solid preparation.

Once formed, the solid CB-183,315 preparations obtained from the sugar solution can be combined with excipients to obtain a pharmaceutical composition formulated for oral delivery (See, for example, Table 1, Formulations Q and U). Examples of oral delivery pharmaceutical compositions include tablets, capsules, sachets or other oral dosing forms.

Solid CB-183,315 preparations (i.e., CB-183,315 (without sugar) or CB-183,315/sugar formulations) were stored for various time periods (e.g., 1-3 months, 1-6 months, 1-12 months) at various temperatures ranges (e.g., 25 and 40 degrees C.), followed by dissolution of the solid preparation and subsequent detection of the amount of CB-183,315 and substances structurally similar to CB-183,315 in the dissolved liquid composition as described in Example 10. Preferred compositions included Formulations M and Q (Example 2 and 6), and Formulations R, S and T (Example 2). Each of these formulations are solid forms of CB-183,315 formed by lyophilizing (Example 9) or spray drying (Example 8) a solution of CB-183,315 and one or more sugars. Table 1 provides a description of examples of solid forms of CB-183,315. Formulation U is a pharmaceutical composition (tablet form for oral administration) comprising the Formulation M and additional excipients, as described in Table 1.

A series of comparative formulations were also prepared, as described in Table 1. The CB-183,315 used in each comparative formulation was not obtained by converting a solution of a sugar and CB-183,315 to a solid. Instead, the CB-183,315 was directly combined with various excipients to form a pharmaceutical formulation suitable for oral delivery. Comparative Formulas A-D were prepared according to Example 1. Comparative Formulation N was prepared according to Example 3, the CB-183,315 material was mixed as a solid with mannitol and other excipients (i.e., the mannitol and the CB-183,315 was not obtained by dissolving CB-183,315 with the mannitol in a solution and converting the solution to a solid). Comparative Formulation O was prepared according to Example 4 by combining CB-183,315 with certain excipients. In Comparative Formulation P, prepared according to Example 5, the CB-183,315 material was mixed as a solid with sucrose and other excipients (i.e., the sucrose and the CB-183,315 was not obtained by dissolving CB-183,315 with the sucrose in a solution and converting the solution to a solid).

TABLE 1

| | Method of | |
|---|---|---|
| Formulation ID | Preparation | Composition (w/w) |
| A | A | 100% CB-183,315, pH 3-4 |
| B | A | 100% CB-183,315, pH 5.0 |
| C | A | 100% CB-183,315, pH 6.0 |
| D | A | 100% CB-183,315, pH 7.0 |
| E | B | 66.7% CB-183,315 |
| | | 33.3% Sucrose |
| | | pH 3-4 |
| F | B | 50% CB-183,315 |
| | | 50% sucrose |
| | | pH 3-4 |
| G | B | 33.3% CB-183,315 |
| | | 67.7% sucrose |
| | | pH 3-4 |
| H | B | 33.3% CB-183,315 |
| | | 67.7% sucrose |
| | | pH 5.0 |
| I | B | 33.3% CB-183,315 |
| | | 67.7% sucrose |
| | | pH 5.5 |
| J | B | 33.3% CB-183,315 |
| | | 67.7% sucrose |
| | | pH 6.0 |
| K | B | 33.3% CB-183,315 |
| | | 67.7% sucrose |
| | | pH 6.5 |
| L | B | 33.3% CB-183,315 |
| | | 67.7% sucrose |
| | | pH 7.0 |
| M | B | 45.45% CB-183,315 |
| | | 54.55% Sucrose |
| | | pH 6.0 |
| N | C | 85% CB-183315, pH 3 |
| | | 12% Mannitol |
| | | 1% Imperial Talc |
| | | 2% Sodium Stearyl Fumarate |
| O | D | 46.97% CB-183,315, pH 7.0 |
| | | 35.79% Microcrytalline |
| | | Cellulose |
| | | 11.49% Mannitol |
| | | 3.00% Croscarmellose Sodium |
| | | 2.00% Stearic Acid |
| | | 0.75% Magnesium Stearate |
| | | 5% Opadry AMB (weight gain |
| | | based on core weight of tablet) |
| P | E | 44.55% CB-183,315, pH 6.0 |
| | | 24.0% Sucrose |
| | | 19.00% Microcrytalline |
| | | Cellulose |
| | | 5.70% Silicon Dioxide |
| | | 5.75% Croscarmellose Sodium |
| | | 1.00% Magnesium Stearate |
| | | 5% Opadry AMB (weight gain |
| | | based on core weight of tablet) |
| Q | F | 85% CB-183,315/Sucrose |
| | | (1:1.1), pH 6.0 |
| | | 3.5% Microcrystalline Cellulose |
| | | 6.0% Silicon Dioxide |
| | | 5% Croscarmellose Sodium |
| | | 0.5% Magnesium Stearate |
| | | 5% Opadry II 85F (weight gain |
| | | based on core weight of tablet) |
| R | B | 50% CB-183,315 |
| | | 50% Trehalose |
| | | pH 6.0 |
| S | B | 50% CB-183,315 |
| | | 25% Trehalose |
| | | 25% Dextran |
| | | pH 6.0 |
| T | B | 50% CB-183,315 |
| | | 50% Trehalose |
| | | pH 2.0 |
| U | G | 71.4% CB-183,315/Trehalose |
| | | (1:1), pH 6.0 |
| | | 11.5% Mannitol |
| | | 11.5% Microcrystalline |
| | | Cellulose |
| | | 4% Polyvinyl Pyrrolidone |
| | | 1% Silicon Dioxide |
| | | 0.6% Magnesium Stearate |

Preferred CB-183,315 solid formulations include formulations selected from
1. 85 weight percent of lyophilized CB-183,315/sucrose, 3.5 weight percent microcrystalline cellulose, 5 weight percent Croscarmellose sodium, 6 weight percent Silicon Dioxide, and 0.5 weight percent Magnesium Stearate, wherein the lyophilized CB-183,315/sucrose is prepared by
a. forming an aqueous solution of the CB-183,315 and sucrose at a ratio of CB-183,315:sucrose of about 1:1.1, at a pH of about 6; and
b. lyophilizing the solution of step (i) to give a lyophilized CB-183,315/sucrose.
2. 85 weight percent of lyophilized CB-183,315/sucrose, 3.5 weight percent microcrystalline cellulose, 5 weight percent Croscarmellose sodium, 6 weight percent Silicon Dioxide, and 0.5 weight percent Magnesium Stearate, wherein the lyophilized CB-183,315/sucrose is prepared by
a. forming an aqueous solution of the CB-183,315 and sucrose at a ratio of CB-183,315:sucrose of about 1:1.1, at a pH of about 6; and
b. spray drying the solution of step (i) to give a lyophilized CB-183,315/sucrose.

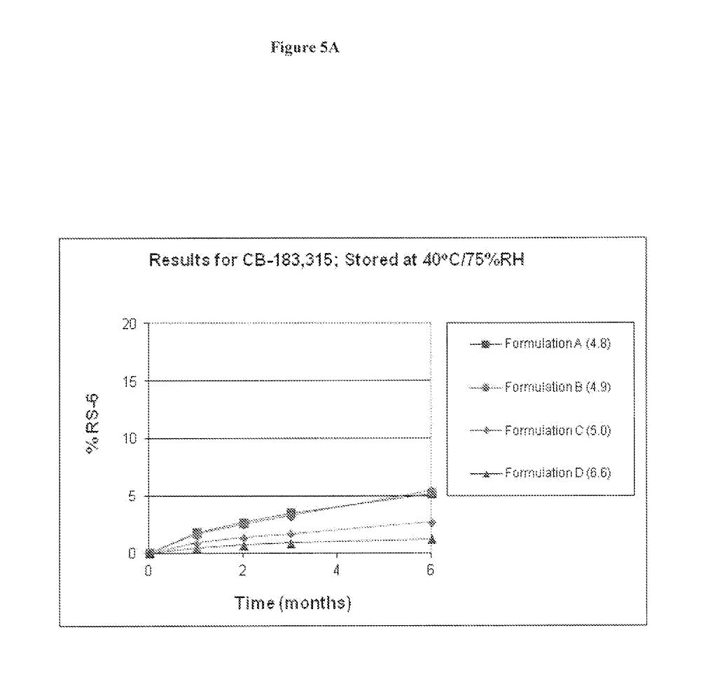

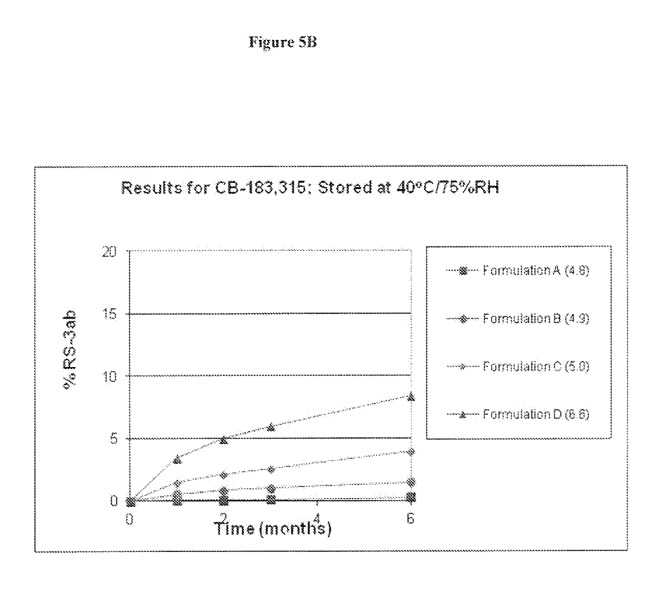

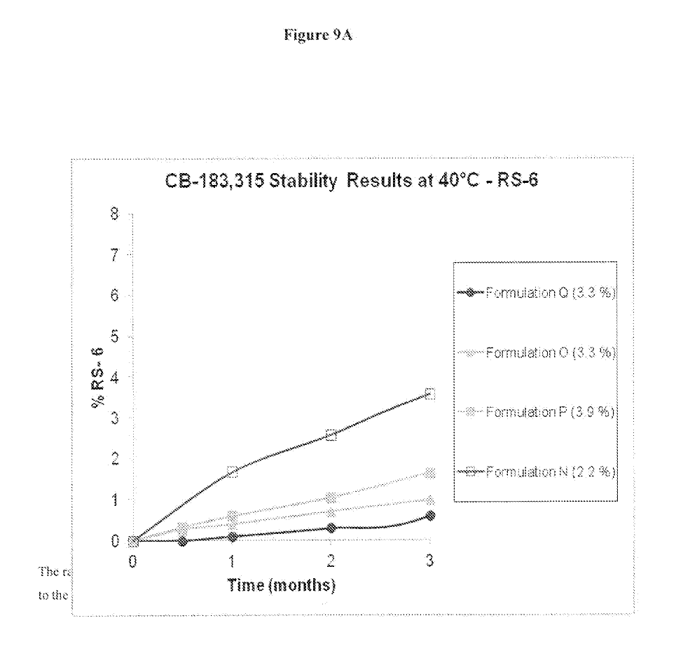

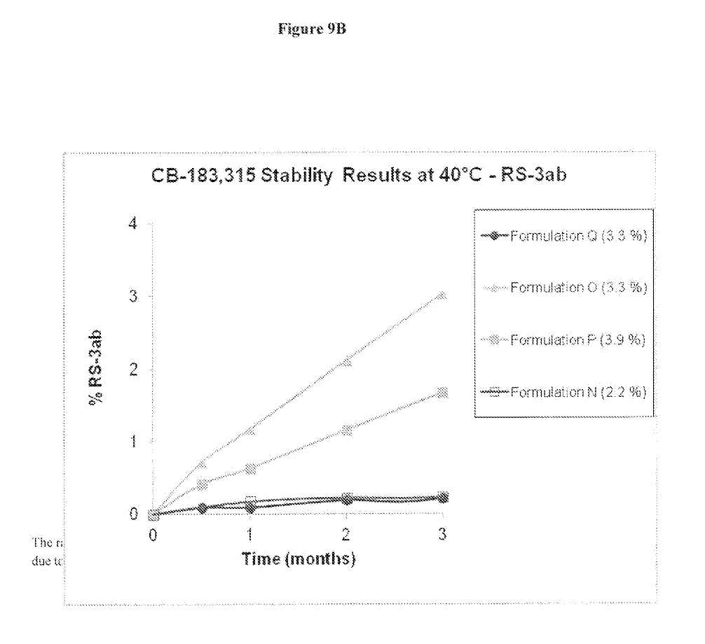

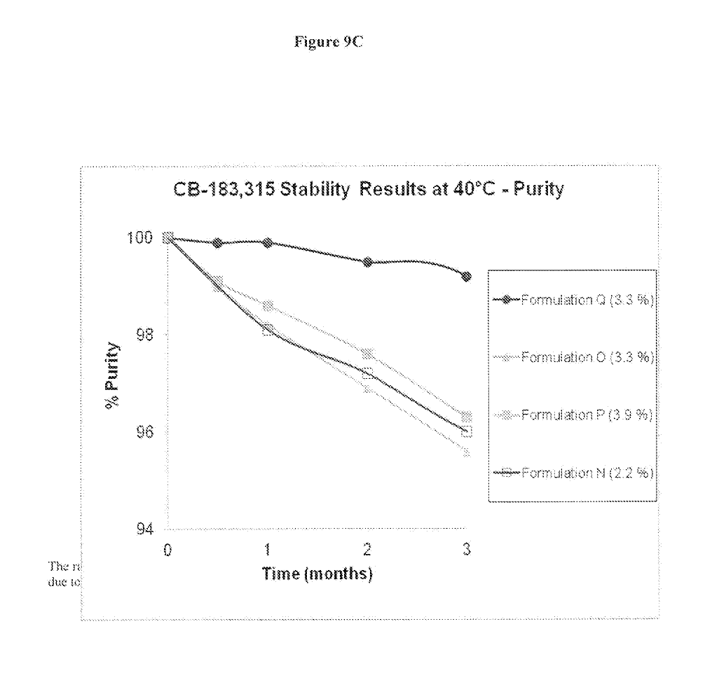

The chemical stability of Formulations in Table 1, including comparative formulations, was measured using the HPLC method in Example 10. It will be understood by one of skill in the art that in FIGS. 5A, 6A, 7A, 8A, 9A, 10A, 11A, 12A, and 13A each data point in the Figure represents a measurement of the percent increase of the RS-6 impurity taken at time periods from 0 to up to 12 months. The chemical stability of each formulation is indicated by the slope of the lines connecting the data points. Similarly for FIGS. 5B, 6B, 7B, 8B, 913, 10B, 11B, 12B, and 13B each data point in the Figure represents a measurement of the percent increase of the RS-3ab impurity taken at time periods from 0 to up to 12 months. The chemical stability of each formulation is indicated by the slope of the lines connecting the data points. Finally for FIGS. 9C, 10C, 11C, 12C, and 13C each data point in the Figure represents a measurement of the percent decrease of the CB-183,315 taken at time periods from 0 to up to 12 months. The chemical stability of each formulation is indicated by the slope of the lines connecting the data points.

Applicants have shown (vide supra) that when in a solid form, the stability of CB-183,315 (no sugar) over time is impacted by the pH level of the CB-183,315 when made (e.g., by lyophilizing or spray dying of the CB-183,315 in solution at a particular pH). FIG. 5A is a graph showing the percent increase of Impurity RS-6 of CB-183,315 preparations (CB-183,315 no sugar) prepared at varying pH measured as a function of time at 40 degrees C. (as described in Example 1). FIG. 5A shows that preparations prepared at low pH (e.g pH≦5, Formulations A and B) show a more rapid increase in the percent of RS-6 impurity when compared to preparations prepared at higher pH (e.g., pH>6, Formulations C and D)

FIG. 5B is a graph showing the percent increase of Impurity RS-3ab of CB-183,315 preparations (CB-183,315 only) prepared at varying pH measured as a function of time at 40 degrees C. (as described in Example 1). FIG. 5B shows that preparations prepared at high pH (e.g pH>6, Formulations C and D) show a more rapid increase in the percent of RS-3ab impurity when compared to preparations prepared at lower pH e.g. pH<5, Formulations A and B).

FIGS. 5A and 5B demonstrate the challenge associated with storing CB-183,315 over time. One of skill in the art will appreciate that stability studies such as those detailed in FIGS. 5A and 5B, conducted over a 6 month period at 40 degrees C., are generally predictive of room temperature stability over a two year period. Therefore, based on the data in FIGS. 5A and 5B, compositions comprising CB-183,315 are not predicted to be stabilized by controlling the pH of the CB-183,315 solution alone to achieve long term shelf life (e.g., 2 years at room temperature).

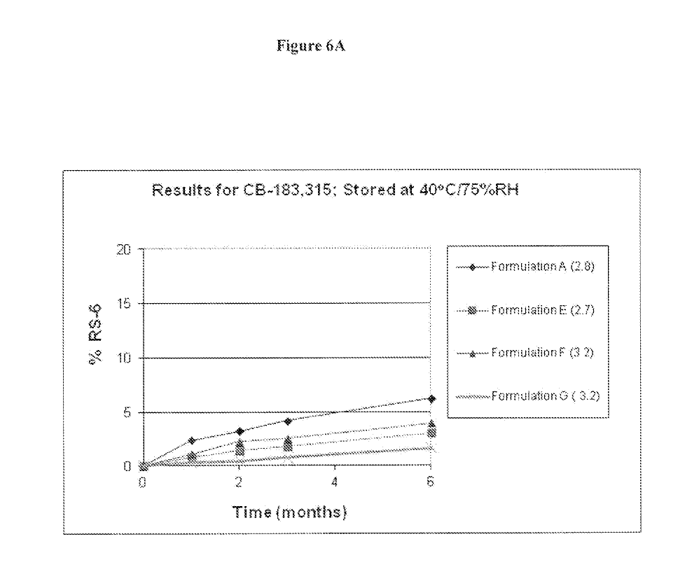

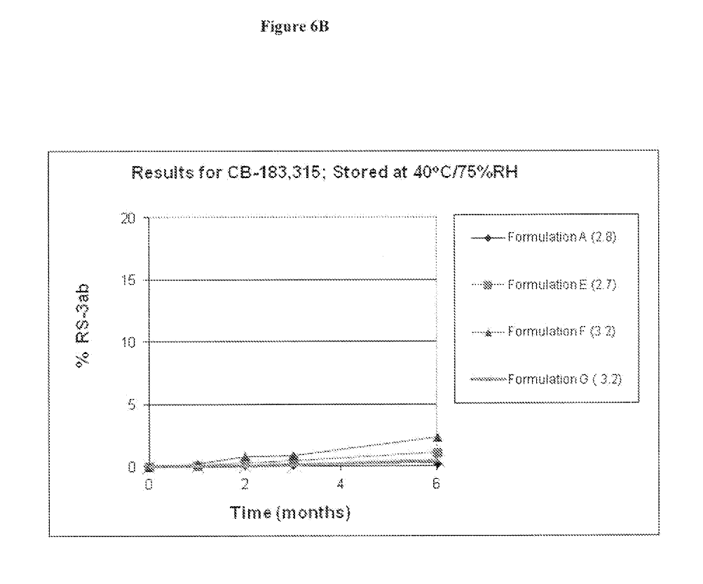

Applicants have discovered that solid compositions of CB-183,315 with increased chemical stability can be achieved when CB-183,315 in solution is combined with one or more sugars (e.g., sucrose or trehalose) and then the solution is converted to a solid form (e.g., by lyophilization or spray drying). As detailed in the graphs in FIGS. 6-13, these novel formulations can negate the pH dependent effect (see FIGS. 5A and B) on the key related substances (RS-6 and RS-3ab) seen in CB-183,315 formulations that are absent the sugar. The graphs and examples below also provide evidence that the CB-183,315/sugar formulations of the invention (i.e., solid pharmaceutical CB-183,315/sugar preparations obtained by converting a solution containing CB-183,315 and a sugar to a solid form) are not only more stable than CB-183,315 (no sugar) formulations, but they are also more stable than compositions in which CB-183,315 is blended as a solid with a sugar (see e.g., Formulations N, O and P).

FIG. 6A is a graph showing the percent increase of Impurity RS-6 in CB-183,315/sucrose formulations formulated at pH 3-4 with varying sucrose concentrations designated Formulation E, F and G and comparative Formulation A (CB-183,315 no sugar) measured as a function of time at 40 degrees C. (as described in Example 10). FIG. 6A shows that over time, CB-183,315/sucrose formulations (Formulations E, F and G) are more stable at low pH and show a slower increase in the amount RS-6 impurity when compared to Formulation A (CB-183,315 (no sugar)). The findings from graphs 6A also suggests that there is a sucrose concentration effect on RS-6 production with the optimal sucrose level at pH 3-4 is in Formulation G.

FIG. 6B is a graph showing the percent increase of Impurity RS-3ab in CB-183,315/sucrose formulations formulated at pH 3-4 with varying sucrose concentrations designated Formulation E, F and G and comparative Formulation A (CB-183,315 no sugar) measured as a function of time at 40 degrees C. (as described in Example 10). FIG. 6B shows that over time, CB-183,315/sucrose formulations (Formulations E, F and G) and comparator Formulation A show little formation of RS-3ab at pH 3-4 which is not surprising as CB-183,315 Formulations were shown to show very slow increase in RS-3ab production at low pH (see graph 5B)

The surprising results from FIGS. 6A and 6B suggest that formulations prepared by combining CB-183,315 in solution with sucrose and then converting the solution to a solid form have a stabilizing effect on RS-6 and RS-3ab production.

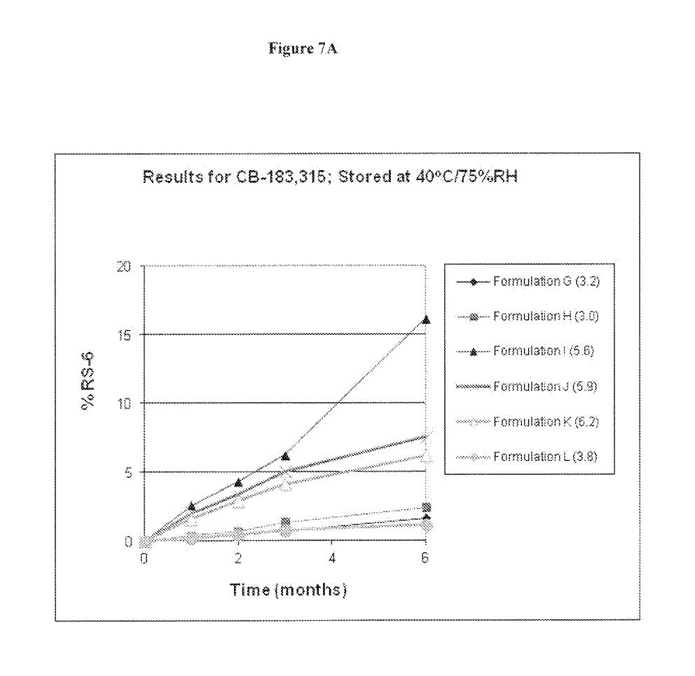

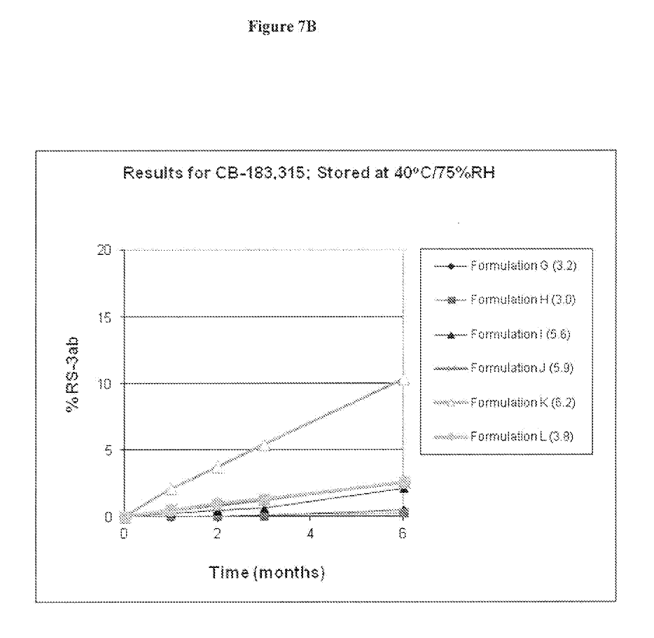

FIG. 7A is a graph showing the percent increase of Impurity RS-6 in CB-183,315/sucrose formulations formulated at identical sucrose concentrations with varying pH designated Formulation G, H, I, J, K and L measured as a function of time at 40 degrees C. (as described in Example 10). The outlier to these data. Formulation I is theorized to be inconsistent due to the high moisture content of this particular sample upon loss of integrity of the container closure for this sampling timepoint.

FIG. 7B is a graph showing the percent increase of Impurity RS-3ab in CB183,315/sucrose formulations formulated at varying pH designated Formulation G, H, I, J, K and L measured as a function of time at 40 degrees C. (as described in Example 10). The outlier to these data, Formulation K is theorized to be inconsistent due to the high moisture content of this particular sample upon loss of integrity of the container closure for this sampling timepoint. FIGS. 7A and 7B suggest that formulations prepared by combining CB-183,315 in solution with sucrose and then converting the solution to a solid form have a stabilizing effect on RS-6 and RS-3ab production across a variety of pH ranges. With the exception of the outliers mentioned, Formulations O, H, I and L display less of an increase of RS-6 and RS-3ab combined than CB-183,315 formulations (no sugar) at similar pH values (see FIGS. 5A and 5B). FIGS. 7A and 7B also suggest that the optimal pH for Formulations comprising 1:1.5 (w/w) CB-183,315 to sugar is about 6.

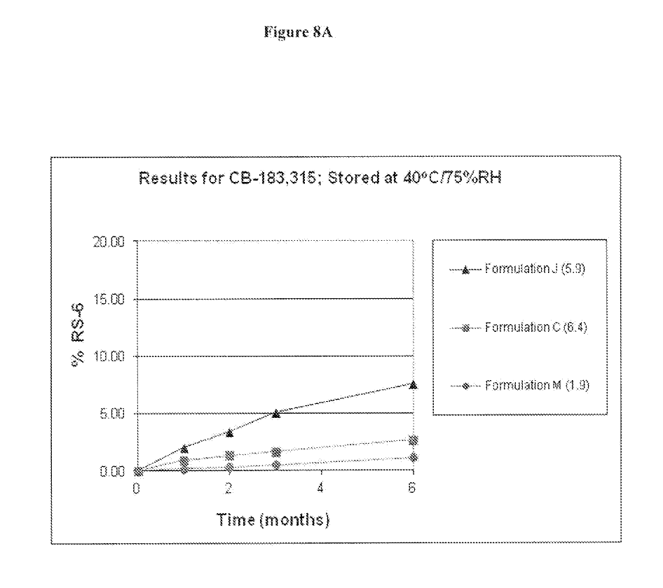

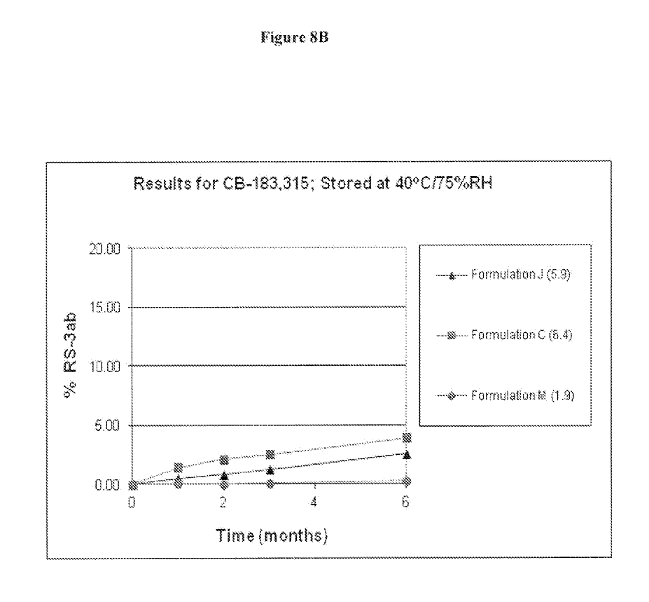

FIG. 8A is a graph showing the percent increase of Impurity RS-6 in CB-183,315/sucrose formulations formulated at pH 6 with varying sucrose concentrations designated Formulations J and M and comparative Formulation A (CB-183,315 no sugar) measured as a function of time at 40 degrees C. (as described in Example 10).

FIG. 8B is a graph showing the percent increase of Impurity RS-3ab in CB-183,315/sucrose formulations formulated at pH 6 with varying sucrose concentrations designated Formulation J and M and comparative Formulation A (CB-183,315 no sugar) measured as a function of time at 40 degrees C. (as described in Example 10). FIGS. 8A and 8B suggests that Formulation M (1:1.14 (w/w) ratio of CB-183,315 to sucrose has the lowest formation of both RS-6 and RS-3ab at pH 6 and represents both the most preferred formula of CB-183,315/sugar, resulting in the lowest increases of both RS-6 and RS-3ab.

FIG. 9A is a graph showing the percent increase of Impurity RS-6 in preferred CB-183,315/sucrose formulation designated Formulation Q and Comparator formulations designated Formulations O, P and N measured as a function of time at 40 degrees C. (as described in Example 10). As noted previously Comparative Formulation N was prepared according to Example 3, the CB-183,315 material was mixed as a solid with mannitol and other excipients (i.e., the mannitol and the CB-183,315 was not obtained by dissolving CB-183,315 with the mannitol in a solution and converting the solution to a solid). Comparative Formulation O was prepared according to Example 4 by combining CB-183,315 with certain excipients. In Comparative Formulation P, prepared according to Example 5, the CB-183,315 material was mixed as a solid with sucrose and other excipients (i.e., the sucrose and the CB-183,315 was not obtained by dissolving CB-183,315 with the sucrose in a solution and converting the solution to a solid). This graph shows that the Formula Q (Formulation Q is a CB-183,315/sucrose, pH 6.0 powder preparation blended with excipients to form a tablet) stabilizes the rate of formation of RS-6 (i.e., there is less RS-6 over time) compared to CB-183,315 (no sugar), pH 6.0 and 7.0 preparations dry blended with sugars (sucrose and mannitol) to form capsules or tablets (Formulations O, P and N). This demonstrates the need to first combine the CB-183,315 and sugar (sucrose) in solution then convert to a solid form (Method B) for further processing into tablets (Method F or G). These results are unexpected based on comparison of the CB-183,315, pH 6.0 alone preparation (see Formulation C, FIG. 5A) which shows higher levels of RS-6 at pH 6.

FIG. 9B is a graph showing the percent increase of Impurity RS-3ab in preferred CB183,315/sucrose formulation designated Formulation Q and comparator formulations designated Formulations O, P and N measured as a function of time at 40 degrees C. (as described in Example 10). This Figure demonstrates that CB-183,315/sucrose preparations blended with excipients to form tablets (e.g., Formulation Q) stabilize the rate of formation of RS-3ab (i.e., there is less RS-3ab over time) at higher pH (pH 6.0) compared to CB-183,315, pH 6.0 and 7.0 preparations dry blended with sugars (sucrose and mannitol) to form capsules or tablets (Formulations O, and P). This demonstrates the need to first combine the CB-183,315 and sugar (sucrose) in solution then convert to a solid form (Method B) for further processing into tablets (Method F or G). These results are unexpected based on comparison of the CB-183,315, pH 6.0 alone preparation (see Formulation C, FIG. 5B) which shows higher levels of RS-3ab at pH 6.

FIG. 9C is a graph showing the percent decrease of CB-183,315 in preferred CB-183,315/sucrose formulation designated Formulation Q and comparator formulations designated Formulations O, P and N measured as a function of time at 40 degrees C. (as described in Example 10). This Figure demonstrates that CB-183,315/sucrose preparations blended with excipients to form tablets (e.g., Formulation Q) stabilize the overall total purity compared to CB-183,315, pH 6.0 and 7.0 preparations dry blended with sugars (sucrose and mannitol) to form capsules or tablets (Formulations O, and P). This demonstrates the need to first combine the CB-183,315 and sugar (sucrose) in solution then convert to a solid form (Method B) for further processing into tablets (Method F or G). These results are unexpected based on comparison of the CB-183,315, pH 6.0 alone preparation (see Formulation C, FIGS. 5A and 5B) and the dry blending of the sucrose with CB-183,315 to form tablets.

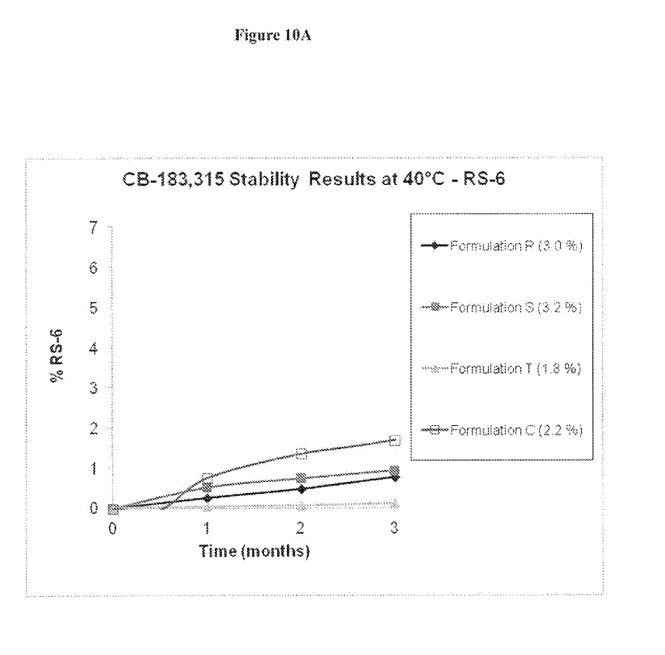

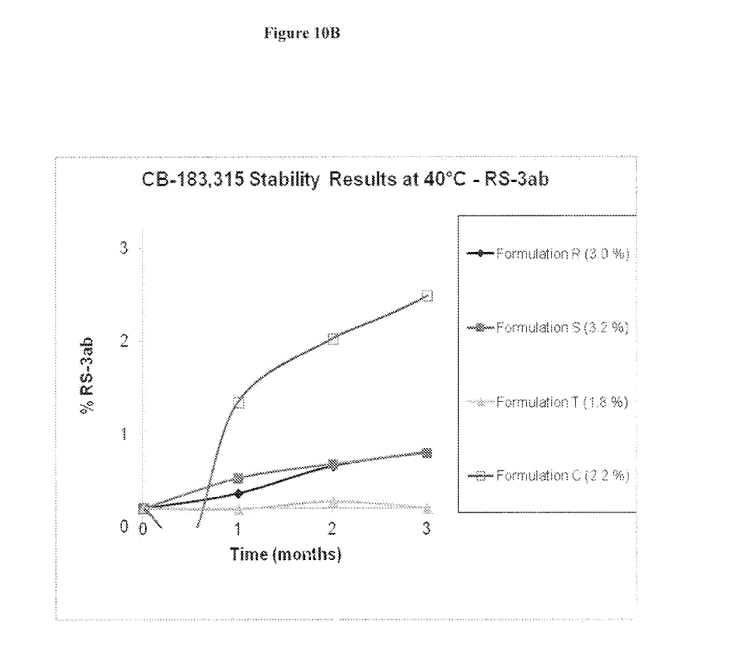

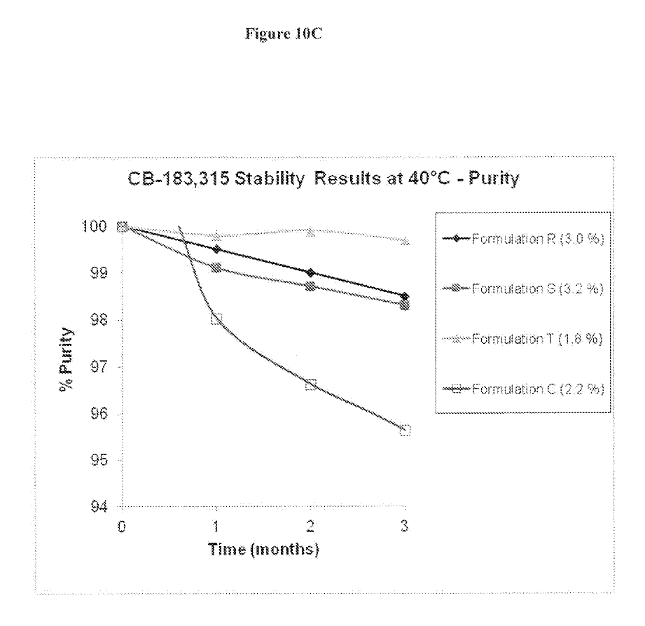

FIG. 10A is a graph showing the percent increase of Impurity RS-6 in CB-183,315/sucrose formulations designated Formulations R, S and T and Comparator formulation designated Formulation C measured as a function of time at 40 degrees C. (as described in Example 10). CB-183,315/trehalose, pH 6.0 (Formulation R) and CB-183,315/trehalose/dextran, pH 6.0 (Formulation 5) and CB-183,315/trehalose, pH 2.0 (Formulation T) powders alone or blended with excipients to form tablets stabilize RS-6 compared to the CB-183,315, pH 6.0 to demonstrate the stabilizing effect of sucrose at higher pH stored at 40° C.

FIG. 10B is a graph showing the percent increase of Impurity RS-3ab in CB183,315/sucrose formulations designated Formulations R, S and T and Comparator formulation designated Formulation C measured as a function of time at 40 degrees C. (as described in Example 10). CB-183,315/trehalose, pH 6.0 (Formulation R) and CB-183,315/trehalose/dextran, pH 6.0 (Formulation S) and CB-183,315/trehalose, pH 2.0 (Formulation T) powders alone or blended with excipients to form tablets stabilize the rate of formation of RS-3ab compared to CB-183,315, pH 6.0 to demonstrate the stabilizing effect of sucrose at higher pH stored at 40° C.

FIG. 10C is a graph showing the percent decrease of CB-183,315 in CB-183,315/sucrose formulations designated Formulations R, S and T and Comparator formulations designated Formulation C measured as a function of time at 40 degrees C. (as described in Example 10). CB-183,315/trehalose, pH 6.0 (Formulation R) and CB-183,315/trehalose/dextran, pH 6.0 (Formulation S) and CB-183,315/trehalose, pH 2.0 (Formulation T) powders alone or blended with excipients to form tablets result in overall higher purity over time compared to CB-183,315, pH 6.0 to demonstrate the stabilizing effect of sucrose at higher pH stored at 40° C.

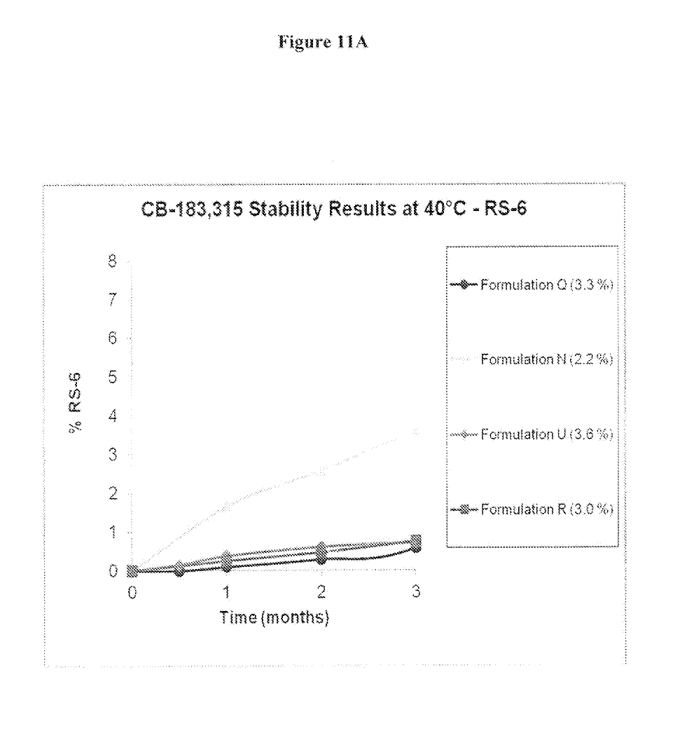

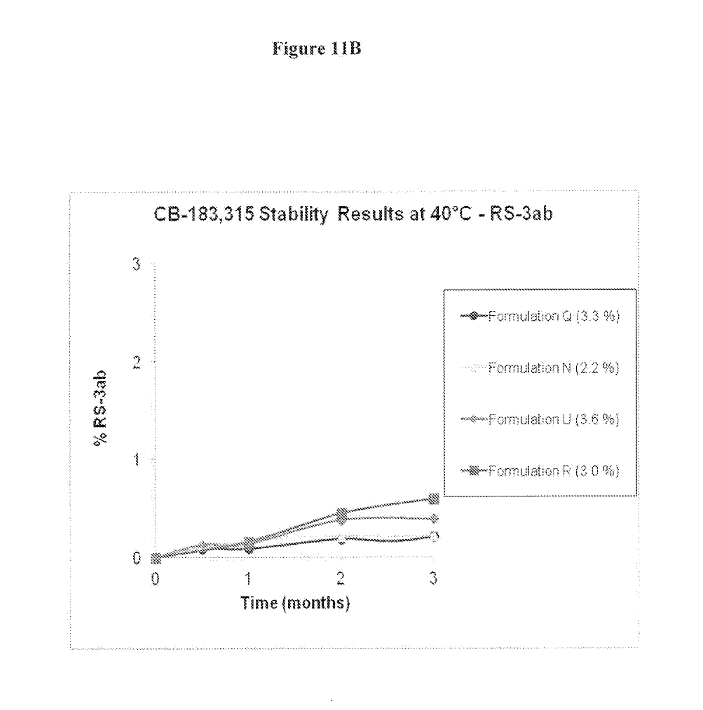

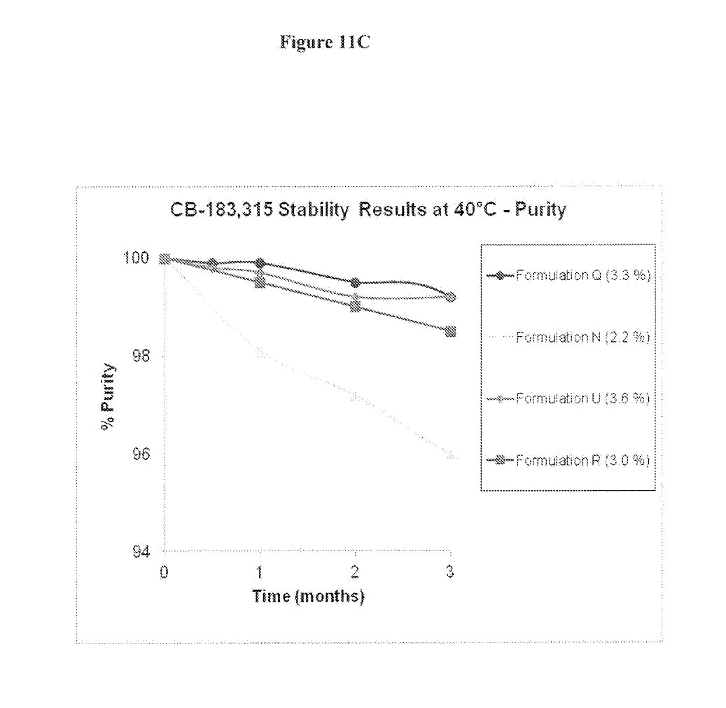

FIG. 11A is a graph showing the percent increase of Impurity RS-6 in preferred CB-183,315/sucrose or trehalose formulations designated Formulations Q (sucrose tablet), U (trehalose tablet) and R (trehalose powder) and Comparator formulation designated Formulation N measured as a function of time at 40 degrees C. (as described in Example 10). This figure shows that at a higher pH plus addition of sucrose (Formulation Q-tablet) or trehalose (Formulations R-powder and U-tablet) combined with CB-183,315 in solution to form a powder stabilizes RS-6 compared to the CB-183,315, pH 3.0 powder (Formulation N-powder) regardless of whether or not the CB-183,315/sugar is in a tablet or powder form.

FIG. 11B is a graph showing the percent increase of Impurity RS-3ab in preferred CB-183,315/sucrose formulations designated Formulations Q (sucrose tablet), U (trehalose tablet) and R (trehalose powder) and Comparator formulation designated Formulation N measured as a function of time at 40 degrees C. (as described in Example 10). The CB-183,315/sucrose or trehalose; pH 6.0 powders blended with excipients then tableted (Formulations Q and U) are as stable as the CB-183,315 alone blended with excipients (Formulation N, Method C) for encapsulation or tableting. CB-183,315/sucrose or trehalose, pH 6.0 powders control the rate of formation of RS-3ab compared to CB-183,315, pH 3.0 alone (Formulation N) which is unexpected at the higher pH of 6.0. In other words, at higher pH CB-183,315 (no sugar) has a high rate of formation of RS-3ab (FIG. 5B), but at a similar pH, the CB-183,315/sugar formations (Formulations Q, U, and R) have a low rate of formation of RS-3ab. Thus FIG. 11B demonstrates the stabilizing effect of sucrose and trehalose at higher pH for RS-3ab.

FIG. 11C is a graph showing the percent decrease of CB-183,315 in preferred CB-183,315/sucrose formulations designated Formulations Q (sucrose tablet), U (trehalose tablet) and R (trehalose powder) and Comparator formulation designated Formulation N measured as a function of time at 40 degrees C. (as described in Example 10). Increase in pH plus addition of sucrose or trehalose combined with CB-183,315 in solution to form a powder results in an overall higher total purity compared to CB-183,315, pH 3.0 powder. This demonstrates the need to combine CB-183,315 and sucrose or trehalose in solution prior to conversion to a solid form.

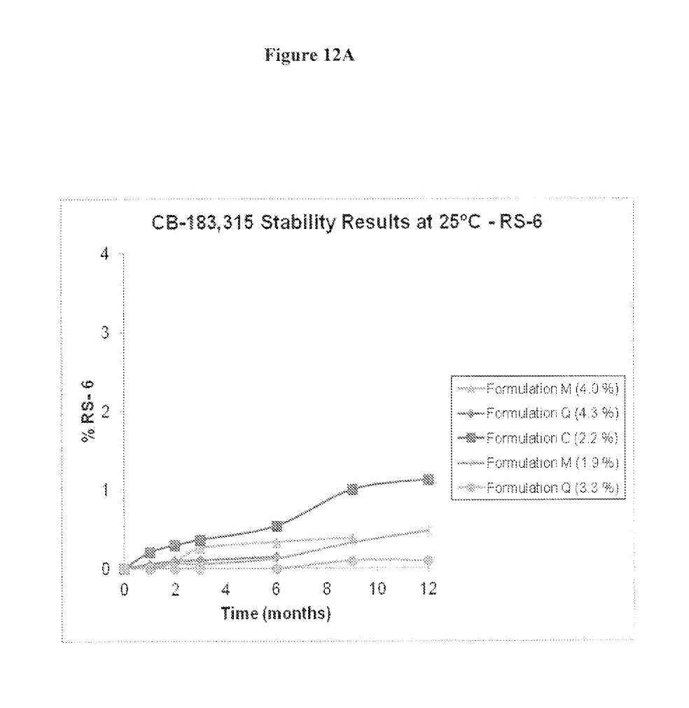

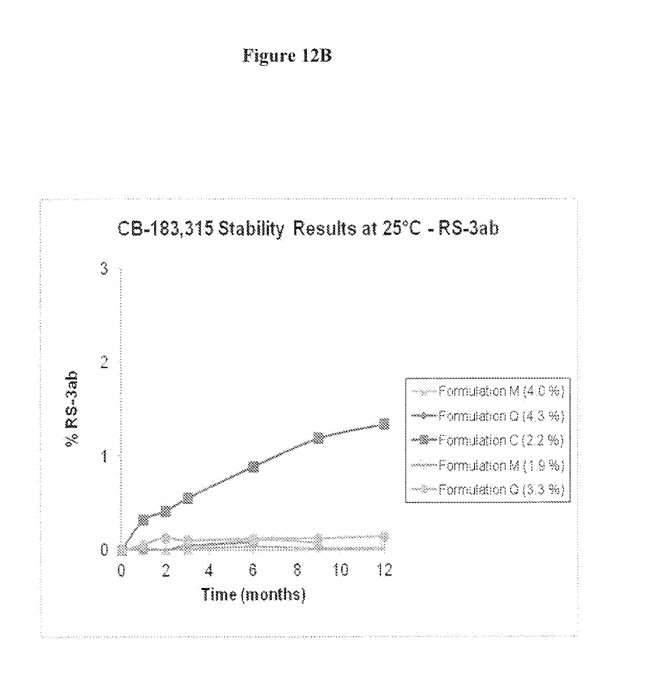

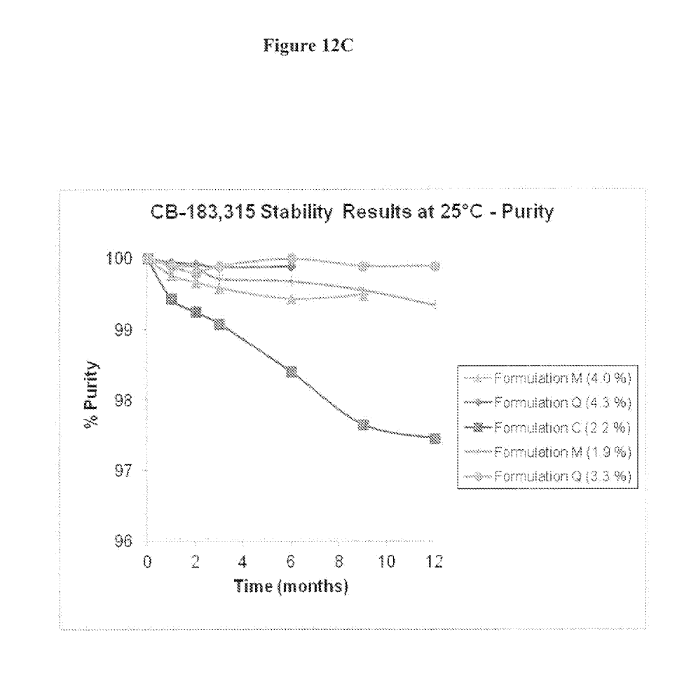

FIG. 12A is a graph showing the percent increase of Impurity RS-6 in preferred formulations designated Formulations Q (tablet) and M (powder) and comparative formulation designated Formula C measured as a function of time at 25 degrees C. (as described in Example 10). CB-183,315/sucrose, pH 6.0 powders alone (Formulation M) and blended with excipients to form tablets (Formulation Q) stabilize the rate of formation of RS-6 compared to the CB-183,315, pH 6.0 powder alone (Formulation C) stored at 25° C., even in the presence of higher moisture contents (Formulations M (4.0%) and Q (4.3%)).

FIG. 12B is a graph showing the percent increase of Impurity RS-3ab in preferred formulations designated Formulations Q and M and comparative formulation designated Formula C measured as a function of time at 25 degrees C. (as described in Example 10)). CB-183,315/sucrose, pH 6.0 powders alone (Formulation M) and blended with excipients to form tablets (Formulation Q) stabilize the rate of formation of RS-3ab, even at higher pH (pH 6.0) which is unexpected based on comparison of the CB-183,315, pH 6.0 alone preparation (Formulation C). This is true even in the presence of CB-183,315/sucrose, pH 6.0 powder preparations containing higher moisture (Formulations M (4.0%) and Q (4.3%)).

FIG. 12C is a graph showing the percent decrease of CB-183,315 in preferred formulations designated Formulations Q and M and comparative formulation designated Formula C measured as a function of time at 25 degrees C. (as described in Example 10). CB-183,315/sucrose, pH 6.0 powders alone (Formulation M) and blended with excipients to form tablets (Formulation Q) result in overall higher total purity levels over time compared to CB-183,315, pH 6.0 powder preparations alone, even in the presence of higher moisture content (Formulations M (4.0%) and Q (4.3%)) stored at 25° C.

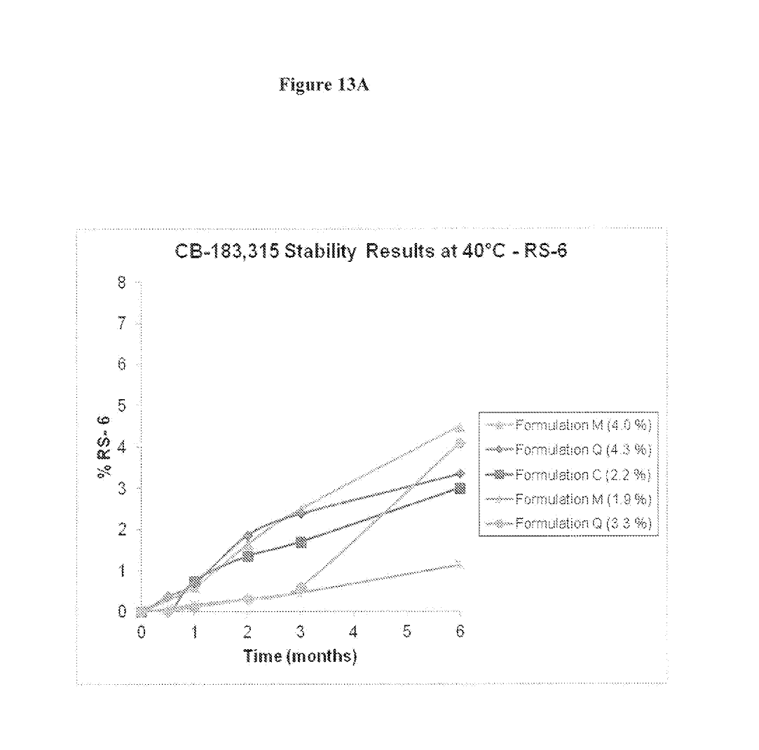

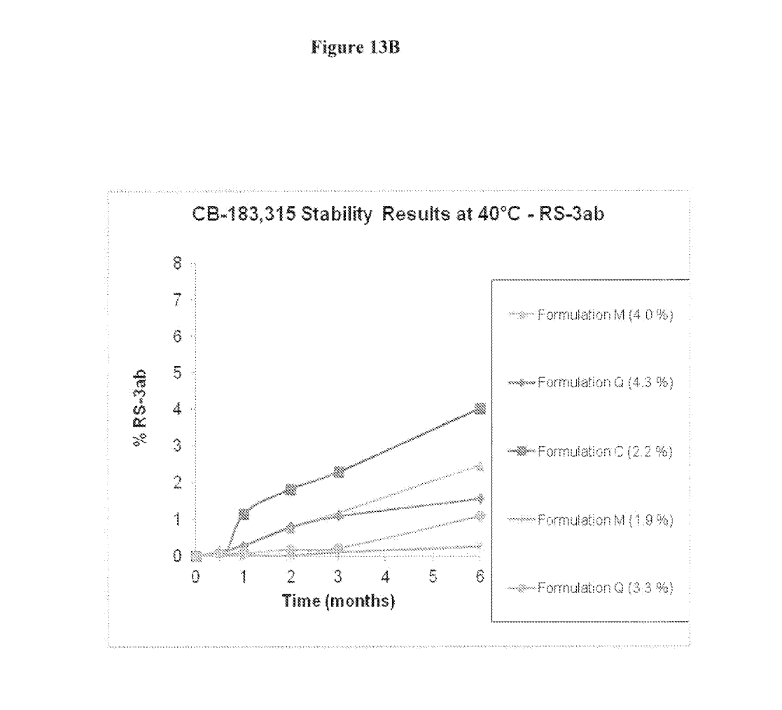

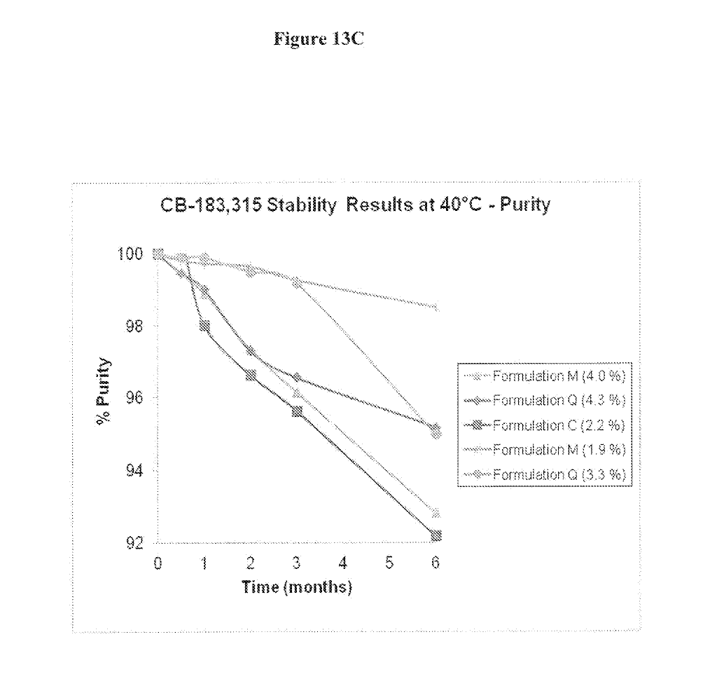

FIG. 13A is a graph showing the percent increase of Impurity RS-6 in preferred formulations designated Formulations Q and M and comparative formulation designated Formula C measured as a function of time at 40 degrees C. (as described in Example 10). CB-183,315/sucrose, pH 6.0 powders alone (Formulation M) and blended with excipients to form tablets (Formulation Q) stabilize the rate of formation of RS-6 compared to the CB-183,315, pH 6.0 powder alone stored at 40° C. however, the rate of formation of RS-6 in the presence of higher moisture contents (Formulations M (4.0%) and Q (4.3%)) at elevated temperature results in similar or unaffected degradation rates compared to the CB-183,315, pH 6.0 powder alone (Formulation C). Of note, Formulation Q (3.3%) tablet packaging integrity of the stability sample may have been compromised causing the sudden increase in RS-6 levels between the 3 & 6 month time-point.

FIG. 13B is a graph showing the percent increase of Impurity RS-3ab in preferred formulations designated Formulations Q and M and comparative formulation designated Formula C measured as a function of time at 40 degrees C. (as described in Example 10). CB-183,315/sucrose, pH 6.0 powders alone (Formulation M) and blended with excipients to form tablets (Formulation Q) stabilize the rate of formation of RS-3ab, even at higher pH (pH 6.0) which is unexpected based on comparison of the CB-183,315, pH 6.0 alone preparation. This is true even in the presence of CB-183,315/sucrose, pH 6.0 powder preparations containing higher moisture (Formulations M (4.0%) and Q (4.3%)) stored at accelerated temperature conditions (40° C.).

FIG. 13C is a graph showing the percent decrease of CB-183,315 in preferred formulations designated Formulations Q and M and comparative formulation designated Formula C measured as a function of time at 40 degrees C. (as described in Example 10). CB-183,315/sucrose, pH 6.0 powders alone (Formulation M) and blended with excipients to form tablets result in overall higher total purity levels over time compared to CB-183,315, pH 6.0 powder stored at 40° C., however, the overall total purity in the presence of higher moisture contents (Formulations M (4.0%) and Q (4.3%)) at elevated temperature results in similar or unaffected degradation rates compared to the CB-183,315, pH 6.0 powder alone (Formulation C).

Of note, Formulation Q tablet packaging integrity of the stability sample may have been compromised causing the sudden increase in RS-6 levels between the 3 & 6 month time-point.

Collectively, FIGS. 6 through 13 show the unexpected discovery that combining CB-183,315 in solution with one or more sugars (e.g., sucrose or trehalose) and then converting the solution to a solid form (e.g., by lyophilization or spray drying) provides a solid composition with increased CB-183,315 chemical stability, including pharmaceutical compositions formulated for oral delivery, obtained by combining these solid forms with one or more excipients.

### EXAMPLES

The following examples are illustrative of preferred embodiments of the inventions described herein.

Solid CB-183,315/sugar preparations were obtained by (a) forming a solution containing CB-183,315 and one or more sugars (e.g., at a pH of about 2-7), and (b) converting the solution to a solid preparation (e.g., by lyophilizing or spray drying). In some examples, the solid preparation (step b) was combined with excipients according to one of several methods to form tablets containing certain preferred pharmaceutical compositions.

The Examples disclose improved CB-183,315 stability in solid pharmaceutical preparations prepared by combining CB-183,315 in solution with one or more sugars and then converting the solution to a solid form. For instance, CB-183,315/sugar formulations listed in Table 1 showed lower percent decrease of CB-183,315 (i.e., higher purity) over a 3-12 month period of time period compared to comparative CB-183,315 (no sugar) in Table 1. Stability of CB-183,315/sucrose in solid formulations was measured relative to the anhydro isomer of CB-183,315 (RS-6, FIG. 3) and the mixture of co-eluted compounds, beta-isomer of CB-183,315 (FIG. 2) and RS-3a (FIG. 4), collectively known as “RS-3ab”, as measured by HPLC.

The present invention will be further understood by reference to the following non-limiting examples. The following examples are provided for illustrative purposes only and are not to be construed as limiting the scope of the invention in any manner.

### Example 1

### Preparation of CB-183,315 Powder: Formulations A-D

### Method A:

CB-183,315 at room temperature was dissolved in chilled water to a concentration of 100 mg/mL. Once the CB-183,315 was dissolved, the solution was pH adjusted by slowly adding chilled 2 N sodium hydroxide or 1N hydrochloric acid until the target pH was achieved. The solution was then lyophilized or spray dried to form a powder (See Examples 8 and 9 below).

### Example 2

### Preparation of CB-183,315/Sucrose Powder: Formulations E, F, G, H, I, J, K, L, M, R, S, and T

### Method B:

CB-183,315 at room temperature was dissolved in chilled water to a concentration of 100 mg/mL. Once the CB-183,315 was dissolved, the appropriate amount of sugar(s) was weighed out and added to the solution. The CB-183,315 solution was mixed until complete dissolution of the sugar(s) was observed. The pH was then adjusted by slowly adding chilled 2 N sodium hydroxide or 1 N hydrochloric acid until the target pH was achieved. The solution was then lyophilized (Formulations E-M) or spray dried (Formulations R-T) to form a powder. (See Examples 8 and 9 below).

### Example 3

### Preparation of CB-183,315 Comparator Formulation N

### Method C:

The composition for Formulation N are identified in Table 1. CB-183,315 powder at room temperature was compacted by cycling small quantities through a ball mill (Restch Mixer Mill) at 15 Hz for 30 seconds producing a very fine densified powder. The milled drug substance was combined and sieved through a 30 mesh screen to obtain a uniform powder with particle size less than 600 μm.

Required amounts of excipients (mannitol, imperial talc 500 and sodium stearyl fumarate) were sieved through an appropriate sized mesh screen and sequentially blended with the densified CB-183,315 powder using a V-blender. The formulated blended was roller compacted then passed through a 25 mesh screen. The compacted blend was loaded into the V-blender to blend with additional sodium stearyl fumarate for external lubrication purpose. The granulated blend was transferred into Lyoguard® freeze drying trays and dried under vacuum for not less than 10 hours at 35° C. in a freeze dryer. Post drying, the granulated blend was filled into hard gelatin capsules using an automated encapsulator equipped with size 00 capsule handling tooling.

### Example 4

### Preparation of CB-183,315 Comparator Formulations O

### Method D:

Formulation O incorporates high shear mixing with stearic acid and mannitol mixed with CB-183,315 (not lyophilized with sucrose, as in Formulation Q). The material can then be blended, roller compacted, sized, blended and compressed into a tablet. The composition for Formulation O is as defined in Table 1 and the percentages of excipients added intra- and intergranular as detailed in the Table 2.

TABLE 2

| | % |
|---|---|
| Component | Formula |
| CB-183315 | 46.97 |
| Stearic Acid | 2.00 |
| (intra) | |
| Microcrystalline | 15.79 |
| cellulose (intra) | |
| Mannitol (intra) | 11.49 |
| Microcrystalline | 20.00 |
| cellulose (inter) | |
| Croscarmellose, | 3.00 |
| Sodium (inter) | |
| Magnesium | 0.75 |
| Stearate (inter) | |
| Core Total | 100.0 |
| OPADRY amb | 5.00* |
| (coating) | |

*Note: Coating was applied to the tablet core based on the average tablet weights

### Procedure:

The CB-183,315 and stearic acid was co-screened through a #20 mesh screen and added to the high shear mixer and mixed for 20 minutes at an impeller speed of 350 rpm and chopper speed of 1500 rpm. The contents were discharged from the mixer then added into the V-blender. The microcrystalline cellulose and mannitol were added and blended for 5 minutes. The resulting blend was then roller compacted and passed through an oscillating mill equipped with a mesh screen. The milled material was then added to the V-blender. The intergranular croscarmellose sodium and microcrystalline cellulose was added to the V-blender and blended for 5 minutes at a suitable rate. Half of the blend material was removed from the V-blender, transferred into a bag and bag blended with the intergranular magnesium stearate then passed through a 20 mesh hand screen. The bag blended material was added back to the V-blender and blended for 3 minutes at suitable rate.

The granulated blend was then charged into the hopper of the tablet press. Tablets were compressed to a target weight of 650 mg. Upon completion of tablet compression, a 20% suspension of coating was prepared by adding approximately 100 g solids to 400 g of purified water. Coating was applied in a pan coater until 5% weight gain to the average tablet core weight was achieved.

### Example 5

### Preparation of Comparative Formulation P

### Method E:

Formulation P incorporates high shear mixing with silicon dioxide and sucrose mixed with CB-183,315 (not lyophilized with sucrose, as in Formulation Q). The material can then be blended, roller compacted, sized, blended and compressed into a tablet. The composition for Formulation P is as defined in Table 1 and the percentages of excipients added intra- and intergranular as detailed in the Table 3.

TABLE 3

| | % |
|---|---|
| Component | Formula |
| CB-183315 | 44.55% |
| Silicon | 5.70% |
| Dioxide(intra) | |
| Sucrose (intra) | 24.00% |
| Croscarmellose, | 2.85% |
| Sodium (intra) | |
| Magnesium | 0.50% |
| Stearate (intra) | |
| Microcrystalline | 19.00% |
| Cellulose (inter) | |
| Croscarmellose, | 2.90% |
| Sodium (inter) | |
| Magnesium | 0.50% |
| Stearate (inter) | |
| Core Total | 100.0 |
| OPADRY amb* | 5.00 |
| (coating) | |
| | N/A |

*Note: Coating was applied to the tablet core based on the average tablet weights

The CB-183,315, silicon dioxide and sucrose was co-screened through a #20 mesh hand screen and mixed in the high shear mixer for 20 minutes at impeller speed of 350 rpm and chopper speed of 1500 rpms. The content was discharged from the mixer then transferred into the V-blender. The croscarmellose sodium was then added and blended for 5 minutes. Half the amount of blend material was removed from the blender and transferred into a bag then blended with magnesium stearate (intra), co-screen through #20 mesh screen and added back to the V-blender and blended for 3 minutes. The resulting blend was roller compacted then passed through an oscillating mill equipped with a x-mesh screen. The granulated/milled material was transferred to the V-blender. The amount of intergranular croscarmellose sodium and microcrystalline cellulose was adjusted and based on the amount of granulated material and blended for 5 minutes at an appropriate rate. Half of the blend material was removed from the V-blender, transferred into a bag and bag blended with the intergranular magnesium stearate then passed through a 20 mesh hand screen. The bag blended material was added back to the V-blender and blended for 3 minutes at suitable rate.

The granulated blend was then charged into the hopper of the tablet press. Tablets were compressed to a target weight of 650 mg. Upon completion of tablet compression, a 20% suspension of coating was prepared by adding approximately 100 g solids to 400 g of purified water. Coating was applied in a pan coater until 5% weight gain to the average tablet core weight was achieved.

### Example 6

### Preparation of CB-183,315/Sugar-Formulation Q

### Method F:

Formulation Q utilized a CB-183,315/sucrose powder (“Lyophilized or Spray dried CB-183,315/Sucrose Preparation” as described in Method B) with additional excipients as listed in the Table 4. The resulting material can be blended, roller compacted, sized, blended and compressed into tablets.

TABLE 4

| | | % |
|---|---|---|
| | Component | Formula |
| Batch | Lyophilized | 85.03 |
| (200 g) | Sucrose | |
| | formulated CB- | |
| | 183,315 See | |
| | Example 2 | |
| | Silicon Dioxide | 6.00 |
| | M5P | |
| | Croscarmellose | 5.00 |
| | Sodium | |
| | Microcrystalline | 3.47 |
| | Cellulose | |
| | Magnesium | 0.50 |
| | Stearate | |
| | Core Total | 100.0 |
| | OPADRY II 85F | 5.00 |
| | White (coating) | |
| | | N/A |

*Note: Coating was applied to the tablet core based on the average tablet weights

The CB-183,315/Sucrose powder (Formulation M) and silicon dioxide was charged into the V-Blender and blended for 5 minutes. The resultant blend was passed through an Oscillating mill equipped with a 20 mesh screen. The screened material is added back to the V-blender and blended for 5 minutes. Half the amount of blend was removed and transferred into a bag and bag blended with Croscarmellose Sodium and microcrystalline cellulose. The blended material was then passed through a #20 mesh screen and blended for 10 minutes. The blended material was granulated using a roller compactor and the resulting material was passed through an oscillating mill equipped with 20 mesh screen and transferred back to the V-blender. The amount of extra-granular magnesium stearate was adjusted based upon the weight of the granulated/milled material. Half the blend was removed and bag blended with the Magnesium Stearate then screened through a 20 mesh hand screen. The material was added to the V-Blender and blended for 3 minutes.

The granulated blend was then charged into the hopper of the tablet press. Tablets were compressed to a target weight of 700 mg. Upon completion of tablet compression, a 20% suspension of coating was prepared by adding approximately 100 g solids to 400 g of purified water. Coating was applied in a pan coater until 5% weight gain to the average tablet core weight was achieved.

### Example 7

### Preparation of CB-183,315/Sugar Formulation U

### Method G:

Formulation U is a tablet formulation comprising Formulation R (Method B) and additional excipients. Formulation U was prepared according to Method B then blended with excipients to form tablets as follows.

The CB-183,315/Trehalose spray dried powder (Formulation R) was added to the appropriate sized container. Microcrystalline cellulose, mannitol, PVP-XL and intragranular colloidal silicon dioxide (screened through a 20 US mesh) was added to the container and blended for 15 minutes at the default mixing speed of the turbula mixer. The magnesium stearate was added to the container (screened through a 20 US Mesh) and blended for 4 minutes at the default mixing speed of the turbula mixer. Using a single station F press, slugs were compressed using the parameters shown in Table 5. Slugs were made by filling the die volume to capacity with the blended and then compressed using the F press to a tensile strength of roughly 0.500 MPA. The slugs were crushed into powder granules using a mortar and pestle then passed through a 20 mesh screen in order to remove smaller particles. Screening of the material and reprocessing using the mortar and pestle was repeated in order to avoid breaking down of the dry granulated particles. Colloidal silicon dioxide (screened through a 20 mesh) was added intragranular and blend for 15 minutes at the default mixing speed of the turbula mixer. Intragranular Magnesium stearate (screened through a 20 US mesh) was added intragranular and blended for 4 minutes at the default mixing speed of the turbula mixer.

Using a single station F press, the Tablets were compressed using the parameters shown in Table 5.

TABLE 5

| Parameter | Value |
|---|---|
| Tooling size | 1.0000 inch, Flat beveled |
| Slug weight | 3392.5 mg (roughly) |
| Tensile Strength | 0.500 MPa |
| Press Setting | 34 |
| Average Slug crushing force | 16.25 kP |
| Average Slug Thickness | 6.6 mm |
| Average main compression force^{b} | 28.7 to 35.1 kN |

### Example 8

### General Procedure for Spray Drying CB-183,315 and CB183,315/Sugar Formulations

The spray dryer was preheated to an outlet temperature of at least 80° C., and the solution (Sec Examples 1-4) was spray dried according to the operating conditions in the table below (Table 6). The spray dried powder was further tray dried in a drying oven for 16 hours.

TABLE 6

| | Mobile Minor in single pass; |
|---|---|
| Spray dryer configuration | 6″ cyclone and 5′ extension |
| Atomizer | Steinen A50 |
| Nozzle Pressure (psig) | 150 |
| Drying Gas Inlet Temperature (° C.) | 180 |
| Drying Gas Outlet Temperature (° C.) | 64 |
| Solution Flow Rate (g/min) | ~40 |
| Drying Gas Flow Rate (g/min) | 1935 |

### Example 9

### General Method for Lyophilization of CB-183,315 and CB-183,315/Sugar Formulations

### Preparation Method:

The CB-183,315 and CB-183,315/sugar solutions (Formulations prepared in Method A and Method B were lyophilized to form a dry powder. The cycle parameters shown in Table 7 were used to form dried powders of Formulations described in Method A and Method B except for preferred Formulation M which was lyophilized according to the cycle parameters shown in Table 8.

TABLE 7
(Methods A & B)

| Step # | Cycle Description |
|---|---|
| 1 | Load product at 5° C. and hold for 60 minutes |
| 2 | Ramp shelf to −50° C. over 180 minutes and hold for 4 hours |
| 3 | Apply vacuum to 90 mTorr and maintain vacuum until |
| | stoppering occurs |
| 4 | Ramp shelf to −15° C. over 6 hours and hold for NLT¹ 40 hours |
| 5 | Ramp shelf to 40° C. over 4 hours and hold for 6 hours |
| 6 | Ramp shelf to 25° C. over 1 hour and hold for 4 hours |
| 7 | Backflush chamber with nitrogen and break vacuum |
| 8 | Product is held at 5° C. until samples are ready for unloading |

¹NLT = not less than

TABLE 8
(Formulation M)

| | | | | Vacuum |
|---|---|---|---|---|
| | Temperature | | | Limit |
| Step | (° C.) | Time (min) | Ramp/Hold | (mTorr) |
| 1 | −30 | 1 | Hold | 150 |
| 3 | −14 | 120 | Hold | 150 |
| 4 | −14 | 4800^{a} | Ramp | 150 |
| 5 | 40 | 180 | Hold | 150 |
| 6 | 40 | 720 | Hold | 250 |
| 7 | 25 | 30 | Ramp | 250 |
| 8 | 25 | 9999 | Hold | 250 |

^{a}Product to remain at Step 3 until primary drying is complete

### Example 10

### Measuring the Amount of CB-183,315 and Substances Structurally Similar to CB-183,315 (e.g., anhydro-CB-183,315 (RS-6), β-isomer of CB-183,315 (RS-3b) and RS-3a, Collectively RS-3ab)

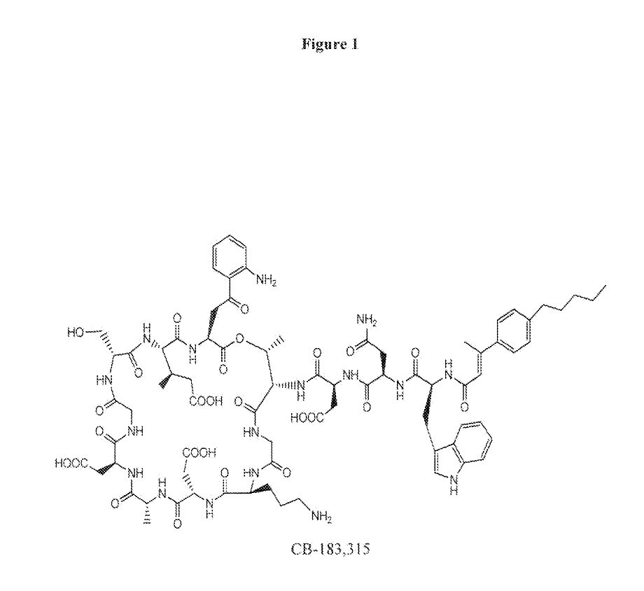

Unless otherwise indicated, the amount of CB-183,315 and three compounds structurally similar to CB-183,315 (FIGS. 1-4) was measured using high performance liquid chromatography (HPLC) analysis in aqueous reconstituted liquid solutions containing CB-183,315, using an Agilent 1100 or 1200 high performance liquid chromatography instrument with an ultraviolet (UV) detector. Peak areas were measured using Waters Empower2 FR5 SPF build 2154 software. Unless otherwise indicated, percent purity of a solid CB-183,315 preparation was determined by reconstituting 20 mg of the solid CB-183,315 preparation in 10 mL of an aqueous diluent to form a reconstituted CB-183,315 solution, then measuring the absorbance of the reconstituted sample at 214 nm by HPLC using the HPLC parameters of Table 3. The percent purity of CB-183,315 in the solid CB-183,315 preparation was calculated by the ratio of absorbance (area under curve) at 214 nm for the CB-183,315 divided by the total area under the curve measured by HPLC of the reconstituted CB-183,315 solution at 214 nm according to Table 3 and the formula below. For a 92% pure CB-183,315 sample, 92% of the total peak area from all peaks≧0.05 area % was attributed to CB-183,315.

In addition, the amount of substances structurally similar to CB-183,315 can be detected by HPLC at 214 nm according to Table 9: anhydro-CB-183,315 (FIG. 3), β-Isomer (FIG. 2) and impurity RS-3a (FIG. 4). Unless otherwise indicated, the amount of these substances in solid CB-183,315 preparations is measured by HPLC according to Table 3 upon reconstitution of 20 mg of the solid CB-183,315 preparation in 10 mL of an aqueous diluent to form a reconstituted CB-183,315 solution, then measuring the absorbance at 214 nm of the reconstituted CB-183,315 by HPLC using the parameters of Table 9.

TABLE 9

| 1. | Solvent Delivery System: | |
|---|---|---|
| | Mode: | Isocratic pumping |
| | Flow rate: | 1.2 mL/min |
| | Run time: | 40 minutes |
| 2. | Solvent A: 50% acetonitrile in 0.45% NH₄H₂PO₄ at pH 3.25 | |
| | Solvent B: 20% acetonitrile in 0.45% NH₄H₂PO₄ at pH 3.25 | |
| | The target condition is approximately 70% Solvent A and 30% | |
| | Solvent B to retain CB-183,315 at 15.0 ± 0.5 minutes; however, | |
| | the solvent ratio may be adjusted to achieve the desired retention | |
| | time. | |
| 3. | Autosampler cooler: | 5 (2 to 8) ° C. |
| 4. | Injection volume: | 20 μL |
| 5. | Column: | IB-SIL (Phenomenex), C-8-HC, 5μ, |
| | | 4.6 mm × 250 mm |
| 6. | Pre-column: | IB-SIL (Phenomenex), C-8, 5μ, |
| | | 4.6 mm × 30 mm |

| | |
|---|---|
| 7. | Detection wavelength: 214 nm |
| 8. | Column Temperature: 22 (20 to 24) ° C. |
| 9. | Integration: A computer system or integrator capable of |
| | measuring peak area. |
| | The purity of CB-183,315 was calculated based on HPLC data, |
| | calculated as follows: |
| | Area % of individual substances structurally similar to CB-183,315 is |
| | calculated using the following equation: |
| | Area % of CB-183,315 and all substances structurally similar to |
| | CB-183,315 as determined using absorbance at 214 nm |
| | Calculate the area of CB-183,315 and all other peaks ≧0.05 area %, |
| | % area = (Aᵢ/Aₜₒₜ)ₓ 100% |
| | where: |
| | % area = Area % of an individual peak; |
| | Aᵢ = Peak of an individual peak; and |
| | Aₜₒₜ = total sample peak area including CB-183,315. |
| | Area % of total substances structurally similar to CB-183,315 is |
| | calculated as the sum of the individual impurities (other than CB- |
| | 183,315)≧0.05%. |
| | *Calculate the % purity of CB-183,315 in Area % using the following |
| | equation: |
| | % CB-183,315 = 100% − % total substances structurally similar to |
| | CB-183,315. |

1. A solid CB-183,315 preparation comprising CB-183,315 and at least one sugar selected from sucrose, trehalose or dextran, wherein the solid preparation is obtained by
a. forming an aqueous solution of the CB-183,315 and the sugar; and
b. converting the aqueous solution of (a) to the solid preparation.

2. The solid CB-183,315 preparation of claim 1 wherein the CB-183,315 to sugar in step (a) is present in a range of about at least 1:0.5 to about 1:2 by weight.

3. The solid CB-183,315 preparation of claim 1, wherein the aqueous solution of step (a) is converted to the solid preparation in step (b) by lyophilization, spray drying, fluid bed drying or spray layering.

4. The solid CB-183,315 preparation of claim 1, obtained by
a. forming an aqueous solution comprising CB-183,315 and a sugar selected from sucrose or trehalose, wherein the CB-183,315 to sugar is present in a range of about at least 1:0.5 to about 1:2 by weight, at a pH of about 2-7, and
b. converting the aqueous CB-183,315 of step (a) to the solid preparation.

5. A method of manufacturing a solid CB-183,315 preparation comprising
a. forming an aqueous solution comprising CB-183,315 and a sugar selected from sucrose or trehalose wherein the CB-183,315 to sugar is present in a range of about at least 1:0.5 to about 1:2 by weight, at a pH of about 2-7, and
b. converting the aqueous CB-183,315 of step (a) to the solid preparation.

6. A tablet, capsule, sachet or oral dosing form comprising a composition of any of claims 1-4.

7. The tablet, capsule, sachet or oral dosing form of claim 6 further comprising one or more pharmaceutically acceptable excipients, carriers or adjuvents.

8. A solid CB-183,315 preparation comprising:
85 weight percent of lyophilized CB-183,315/sucrose, 3.5 weight percent microcrystalline cellulose, 5 weight percent Croscarmellose sodium, 6 weight percent Silicon Dioxide, and 0.5 weight percent Magnesium Stearate, wherein the lyophilized CB-183,315/sucrose is prepared by
i. forming an aqueous solution of the CB-183,315 and sucrose at a ratio of CB-183,315 to sucrose of about 1:1.1, at a pH of about 6; and
ii. lyophilizing the solution of step (i) to give a lyophilized CB-183,315/sucrose.

9. A solid CB-183,315 preparation comprising:
71.4 weight percent of CB-183,315/Trehalose spray dried material, 11.5 weight percent Mannitol, 11.5 weight percent microcrystalline cellulose, 4 weight percent polyvinyl pyrrolidone, 1 weight percent Silicon Dioxide and 0.6 weight percent Magnesium Stearate, wherein the CB-183,315/Trehalose spray dried material is prepared by
i. forming an aqueous solution of the CB-183,315 and trehalose at a ratio of CB-183,315 to trehalose of about 1:1.1, a pH of about 6; and
ii. spray drying the solution of step (i) to give a spray dried CB-183,315/trehalose.

10. A pharmaceutical composition comprising CB-183,315 and sucrose, wherein the solid preparation is obtained by a process comprising the steps of
a. forming an aqueous solution of the CB-183,315 and sucrose at a pH of about 2-6; and
b. converting the aqueous solution of (a) to a solid preparation; and
c. compounding the solid preparation as the pharmaceutical composition for oral delivery.

11. A solid CB-183,315 preparation comprising:
81-85 weight percent of lyophilized CB-183,315/sucrose, 3.5-7 weight percent microcrystalline cellulose, 5 weight percent Croscarmellose sodium, 1-6 weight percent Silicon Dioxide, and 0.5-1 weight percent Magnesium Stearate, wherein the lyophilized CB-183,315/sucrose is prepared by
i. forming an aqueous solution of the CB-183,315 and sucrose at a ratio of CB-183,315 to sucrose of about 1:1.1, at a pH of about 6; and
ii. lyophilizing the solution of step (i) to give a lyophilized CB-183,315/sucrose.

12. A solid CB-183,315 preparation comprising:
81-85 weight percent of spray dried CB-183,315/sucrose, 3.5-7 weight percent microcrystalline cellulose, 5 weight percent Croscarmellose sodium, 1-6 weight percent Silicon Dioxide, and 0.5-1 weight percent Magnesium Stearate, wherein the lyophilized CB-183,315/sucrose is prepared by
i. forming an aqueous solution of the CB-183,315 and sucrose at a ratio of CB-183,315 to sucrose of about 1:1.1, at a pH of about 6; and
ii. spray drying the solution of step (i) to give a spray dried CB-183,315/sucrose.

13. The composition of claim 10, wherein the aqueous solution having a pH of about 6 is converted to the solid preparation by lyophilization, and the solid preparation is combined with one or more excipients to form the pharmaceutical composition.
